(19) United States
(12) Patent Application Publication
(10) Pub. No.: US 2015/0366821 A1
(43) Pub. Date: Dec. 24, 2015

(54) ADRENERGIC AGONISTS FOR USE IN TREATING LIVER DAMAGE

(71) Applicant: UCL BUSINESS PLC, London (GB)

(72) Inventor: Jude Oben, London (GB)

(73) Assignee: UCL Business PLC, London (GB)

(21) Appl. No.: 14/442,075

(22) PCT Filed: Oct. 31, 2013
(86) PCT No.: PCT/GB2013/052843

§ 371 Date: May 11, 2015

Related U.S. Application Data
(60) Provisional application No. 61/725,846, filed on Nov. 13, 2012.

(51) Int. Cl. A61K 31/137; A61K 31/4174; C12N 5/071; A61K 31/4168

(52) U.S. Cl. CPC A61K 31/137; A61K 31/4168; A61K 31/4174; C12N 5/0672

(57) ABSTRACT

The invention relates to liver damage, and to pharmaceutical compositions for use in treating, preventing or ameliorating liver damage or disease, especially acute liver damage. The invention is particularly, although not exclusively, concerned with treating or preventing liver damage caused by paracetamol poisoning. The invention also extends to methods of treating such conditions.

The invention relates to liver damage, and to pharmaceutical compositions for use in treating, preventing or ameliorating liver damage or disease, especially acute liver damage. The invention is particularly, although not exclusively, concerned with treating or preventing liver damage caused by paracetamol (also known as acetaminophen) poisoning. The invention also extends to methods of treating such conditions.

Paracetamol (Acetaminophen, APAP) overdose, either deliberate, through suicide attempts, or unintentionally, because of consumption of multiple-drug preparations containing APAP, is a major public health problem worldwide because it causes much morbidity which frequently progresses to fulminant liver failure (FLF). This is despite the presence of N-Acetyl Cysteine (NAC) as an antidote, and Governmental attempts to reduce the non-prescription availability of APAP. FLF may result in death if a suitable liver for transplantation cannot be found, with about 200 such deaths per year, in England and Wales alone. Besides these deaths, there is the fiscal cost of liver transplantation and subsequent maintenance of these transplanted patients, with total such costs having being estimated worldwide at billions of dollars per year. A shortage of donor livers for transplantation as a treatment for liver disease, including FLF, drives the search to understand the factors that regulate liver regeneration.

There is therefore a need to provide an improved means of treating liver disease or damage. The inventors have surprisingly demonstrated that the activation of α-adrenergic receptors and/or β-adrenergic receptors, which are present on hepatic progenitor cells (stem cells, HPC), promotes the expansion of these stem cells and can therefore be used to treat liver damage.

Thus, in a first aspect of the invention, there is provided an adrenergic receptor agonist, for use in treating, preventing or ameliorating liver damage.

In a second aspect, there is provided a method of treating, ameliorating or preventing liver damage in a subject, the method comprising administering, to a subject in need of such treatment, a therapeutically effective amount of an adrenergic receptor agonist.

Hepatic progenitor cells (HPC) are bi-potential liver resident stem cells that can differentiate into hepatocytes or bile duct cells. They are activated to promote hepatic regeneration and replace lost liver tissue after acute massive hepatocyte loss or when mature hepatocyte replication is impaired, as in chronic liver inflammatory conditions, such as non-alcoholic steatohepatitis. Emerging evidence suggests that the sympathetic nervous system (SNS) may be involved in liver repair, either directly or through effects on liver cells, such as myofibroblastic hepatic stellate cells (HSC), which are regulated positively by the SNS. Also, it has previously been shown the al-adrenoceptor antagonist, prazosin (PRZ), expanded liver progenitors and reduced injury in a chronic model of liver disease.

Therefore, in a further series of experiments with the β-adrenergic receptor antagonist propranolol (PRL), the inventor's starting hypothesis was that the homeostatic effect of SNS signalling on HPC expansion is inhibitory and that PRL would, as with PRZ, expand HPC numbers and reduce liver injury. Initial results, under in vivo conditions simulating non-alcoholic steatohepatitis (NASH), showed emphatically that PRL, like PRZ, expanded the HPC population. However, PRL unlike PRZ, significantly increased biochemical and histological markers of liver injury and cell death. Mechanistic studies showed that PRL induced hepatocyte death, as evidenced by increased release of ALT, LDH, TNF-α and FAS ligand, through both the extrinsic and intrinsic apoptotic pathways as judged by upregulation of FAS receptor, caspase-8 proteins, and cytochrome C. These PRL results caused the inventors to modify their working hypothesis and, as a result, they postulated that surprisingly, the basal action of SNS agonist signalling in liver injury may be to promote HPC expansion.

This modified hypothesis was supported by the finding that infusion of the SNS agonists Norepinephrine (NE) and Isoprenaline (ISO) into spontaneously steatohepatitic ob/ob mice induced increases in HPC number, and a parallel reduction in liver injury. Furthermore, in the complete absence of the SNS in mice lacking Dopamine β-hydroxylase (Dbh^{-/-}) and which therefore cannot synthesize SNS neurotransmitters, a diet inducing NASH led to a loss of the hepatomegaly and expansion of HPC normally associated with this diet and observed in the controls. This reduction was reversed by infusion of ISO. Moreover, although HPC are acknowledged to play only a minor role in liver regeneration after a partial hepatectomy, in the absence of agents that inhibit replication of mature hepatocytes, the inventors surprisingly also observed a clear reduction in HPC numbers in the Dbh^{-/-} mice post hepatectomy. These surprising results suggested unequivocally and for the first time, that direct SNS agonist signalling is required to expand the HPC compartment after acute and chronic liver injury.

In support of the above there is evidence showing that the SNS regulates stem cell physiology in other organs, such that pharmacological manipulation of the SNS has been shown to modulate haematopoietic stem cell proliferation and egress. Moreover, adrenergic agents have been shown to induce proliferation of neuronal stem cells and embryonic stem cells have also been shown to respond to adrenergic stimulation. Given the clinical importance of APAP poisoning and evidence suggesting that the SNS regulates HPC and reduces liver injury, the inventors hypothesized that SNS stimulation by ISO would expand the HPC population and reduce acute liver injury induced by APAP. They also sought to investigate the mechanisms through which ISO affected HPC. The results comprehensively show that HPC are markedly expanded by the SNS β-adrenoceptor agonist ISO through the β-catenin-Wnt pathway and that ISO drastically reduces APAP induced injury. Since there is a possibility that ISO may cause abnormal cardiac rhythms in patients with acute APAP poisoning, the inventors then sought to determine if the al-adrenoceptor agonist phenylephrine, which may induce less abnormal rhythms, also caused an expansion of HPC with reduced liver injury.

Accordingly, the adrenergic receptor agonist may be used for treating, preventing or ameliorating any kind of liver damage or failure. For example, the agonist may be used to treat fulminant liver failure (FLF). The liver damage which is treated may be acute liver damage. For example, the liver damage may have been caused by administration or consumption of a poison, for example paracetamol (i.e. APAP) or alcohol. The liver damage may have been caused by ingestion of Khat plant, which like APAP, may also cause acute liver failure (ALF).

Adrenergic receptors are metabotopic G-protein coupled receptors (GPCRs) that are activated by catecholamines, especially noradrenaline and adrenaline. These receptors are generally classified as either alpha(α)-adrenoceptors or beta(β)-adrenoceptors. Accordingly, in one embodiment, the adrenergic receptor agonist may be an α-adrenergic receptor agonist. In another embodiment, the adrenergic receptor agonist may be a β-adrenergic receptor agonist.

The term “agonist” can mean a molecule that selectively binds to either the α- or the β-adrenergic receptor to initiate the signal transduction reaction. Preferably, the agonist is operable, in use, to selectively activate the desired adrenergic receptor, i.e. the agonist activates the target adrenoceptor to a greater extent, or at lower doses, than other types of adrenergic receptors.

Alpha-adrenergic receptors may further be characterized as either α₁-adrenoceptors or α₂-adrenoceptors. Therefore, the adrenergic receptor agonist may be either an α₁ or an α₂-adrenergic receptor agonist. Activation of alpha₁-adreonceptors promotes the activation of the G protein, G_{q}, which, in turn leads to the activation of the phospholipase C signaling pathway, whereas activation of α₂ adrenoceptors promotes the activation of the G protein, Gᵢ, which in turn leads to the activation of the adenylate cyclase signaling pathway. Hence, a suitable α₁-adrenergic receptor agonist may be selected from a group consisting of: Noradrenaline, Xylometazoline, Phenylephrine, and Methoxamine.

A preferred α₁-adrenergic receptor agonist is Phenylephrine, as described in Example 7. A suitable α₂-adrenergic receptor agonist may be selected from a group consisting of: Clonidine, Dexmedetomidine, Medetomidine, and Romifidine.

The skilled person will appreciate that α₁-adrenoceptors may be further subcategorized as α₁ₐ-, α_{1c}- or α_{1d}-adrenoceptors. α₂-adrenoceptors may be further subcategorized as α_{2b}- or α_{2c}-adrenoceptors.

Beta-adrenergic receptors may be further characterized, as beta₁-adrenoceptors, beta₂-adrenoceptors or beta₃-adrenoceptors. Therefore, the adrenergic receptor agonist may be a β₁-, a β₂- or a β₃-adrenergic receptor agonist. However, in some embodiments, the agonist may not be a β₃-adrenergic receptor agonist. Stimulation of either of the three β-adrenergic receptors promotes the activation of the G protein, Gₛ, which in turn leads to the activation of the adenylate cyclase signaling pathway. A suitable β₁-adrenergic receptor agonist may be selected from a group consisting of: Dobutamine, Isoprenaline, and Noradrenaline. A preferred β₁-adrenergic receptor agonist is Isoprenaline, as described in the Examples.

A suitable β₂-adrenergic receptor agonist may be selected from a group consisting of: Isoprenaline and Salbutamol. As described in the Examples, these agonists will be useful in the treatment of acute liver disease/damage.

In some embodiments of the invention, it may be desirable to administer an α-adrenoceptor agonist and a β-adrenoceptor agonist simultaneously. For example, an α-adrenergic receptor agonist such as Noradrenaline, Xylometazoline, Phenylephrine, or Methoxamine may be administered together with a β₁-adrenergic receptor agonist such as Dobutamine, Isoprenaline, and Noradrenaline. Preferably, Phenylephrine is administered with Isoprenaline.

Classification of α-adrenoceptors and β-adrenoceptors, and their subtypes, may be achieved by comparing the potency of the catecholamines, isoprenaline, adrenaline and noradrenaline at each of these receptors, and possibly also by determining the type of intracellular signaling pathway which is activated by the action of an agonist at the receptor.

Adrenergic receptor agonists used according to the invention may achieve their functional effect through promoting the expansion of hepatic progenitor cells (HPC's). Although not wishing to be bound by any hypothesis, the inventors believe that adrenoceptor agonists promote expansion/proliferation of hepatic progenitor cells through activation of the HPC Wnt pathway, which leads to the expression of various Wnts. Wnts are a family of signaling proteins which pass signals from receptors found on the surface of cells to their nuclei to regulate gene expression.

Accordingly, the agonist may be operable in use to enhance HPC expansion, preferably by activating the Wnt pathway.

Therefore, in a third aspect, there is provided an adrenergic receptor agonist, for use in inducing the expression of Wnt by hepatic progenitor cells.

Preferably, expression of Wnt 1, 3a, 6 or 10a may be induced by the agonist compared to the level of expression in the absence of the agonist.

The term “expression” can relate to the detection of a Wnt protein in any compartment of the cell (e.g. in the nucleus, cytosol, the Endoplasmic Reticulum or the Golgi apparatus); or detection of the mRNA encoding a Wnt.

It will be appreciated that adrenoceptor agonists according to the invention may be used in a medicament, which may be used in a monotherapy, i.e. use of only an adrenoceptor agonist (e.g. an antibody or a catecholamine) for treating, ameliorating, or preventing acute liver damage/disease. Alternatively, adrenoceptor agonists according to the invention may be used as an adjunct to, or in combination with, known therapies for treating, ameliorating, or preventing acute liver damage/disease. For example, adrenoceptor agonists of the invention may be used in combination with known agents for treating acute liver damage/disease, such N-Acetyl Cysteine etc.

The adrenoceptor agonists according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, or any other suitable form that may be administered to a person or animal in need of treatment. It will be appreciated that the vehicle of medicaments according to the invention should be one which is well-tolerated by the subject to whom it is given.

The composition may comprise liver-targeting means, arranged, in use, to target the adrenoceptor agonist at least adjacent the liver. For example, the adrenoceptor agonist may be formulated within a liposome or liposome suspension, which liposome comprises a ligand which targets the liver. Advantageously, such liver targeting significantly improves delivery of the active agent to the treatment site increasing efficacy.

Medicaments comprising adrenoceptor agonists according to the invention may be used in a number of ways. For instance, oral administration may be required, in which case the adrenoceptor agonists may be contained within a composition that may, for example, be ingested orally in the form of a tablet, capsule or liquid. Compositions comprising adrenoceptor agonists of the invention may be administered by inhalation (e.g. intranasally). Compositions may also be formulated for topical use. For instance, creams or ointments may be applied to the skin, for example, adjacent the treatment site, e.g. the liver.

Adrenoceptor agonists according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with adrenoceptor agonists used according to the invention is required and which would normally require frequent administration (e.g. at least daily injection).

In a preferred embodiment, adrenoceptor agonists and compositions according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the adrenoceptor agonist that is required is determined by its biological activity and bioavailability, which in turn depends on the mode of administration, the physiochemical properties of the adrenoceptor agonist and whether it is being used as a monotherapy or in a combined therapy. The frequency of administration will also be influenced by the half-life of the adrenoceptor agonist within the subject being treated. Optimal dosages to be administered may be determined by those skilled in the art, and will vary with the particular adrenoceptor agonist in use, the strength of the pharmaceutical composition, the mode of administration, and the advancement of the disease being treated. Additional factors depending on the particular subject being treated will result in a need to adjust dosages, including subject age, weight, gender, diet, and time of administration.

Generally, a daily dose of between 0.01 μg/kg of body weight and 0.5 g/kg of body weight of the adrenoceptor agonist according to the invention may be used for treating, ameliorating, or preventing liver damage/disease, depending upon which adrenoceptor agonist is used, e.g. catecholamine or antibody. More preferably, the daily dose of the adrenoceptor agonist is between 0.01 mg/kg of body weight and 500 mg/kg of body weight, more preferably between 0.1 mg/kg and 200 mg/kg body weight, and most preferably between approximately 1 mg/kg and 100 mg/kg body weight.

As discussed in the examples, particularly Examples 6 and 7, the adrenoceptor agonist may be administered before, during or after onset of acute liver disease/damage. For example, the agonist may be administered immediately after a subject has ingested a toxic amount of paracetamol. Daily doses may be given as a single administration (e.g. a single daily injection). Alternatively, the adrenoceptor agonist may require administration twice or more times during a day. As an example, adrenoceptor agonists may be administered as two (or more depending upon the severity of the disease being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of adrenoceptor agonist according to the invention to a patient without the need to administer repeated doses.

In another embodiment, the adrenoceptor agonist may be administered before the onset of liver damage. For example, in cases where a subject is undergoing clinical trials or being treated with a drug which is known to, or likely to, cause acute liver damage (for example an anticancer drug), then it may be advantageous to protect the liver by per-administering the adrenoceptor agonist of the invention.

Known procedures, such as those conventionally employed by the pharmaceutical industry (e.g. in vivo experimentation, clinical trials, etc.), may be used to form specific formulations comprising the adrenoceptor agonist according to the invention and precise therapeutic regimes (such as daily doses of the adrenoceptor agonist and the frequency of administration). The inventors believe that they are the first to describe a pharmaceutical composition for treating acute liver disease/damage, based on the use of the agonist of the invention.

Hence, in a fourth aspect of the invention, there is provided a liver damage treatment composition, comprising an adrenergic receptor agonist and a pharmaceutically acceptable vehicle.

Liver damage or disease which may be treated with the composition may be acute. In addition, the liver disease may be caused by a variety of factors, which can include paracetamol or Acetaminophen (APAP) overdose, alcoholism, or other diseases, such as Malaria. The agonist may comprise an α- or a β-adrenergic receptor agonist. In one embodiment, the agonist may be either an α₁ or an α₂-adrenergic receptor agonist. A suitable α₁-adrenergic receptor agonist may be selected from a group consisting of: Noradrenaline, Xylometazoline, Phenylephrine, and Methoxamine. Preferably, the agonist is Phenylephrine. A suitable α₂-adrenergic receptor agonist may be selected from a group consisting of: Clonidine, Dexmedetomidine, Medetomidine, and Romifidine. In another embodiment, the agonist may be a β₁-, a β₂- or a β₃-adrenergic receptor agonist. A suitable β₁-adrenergic receptor agonist may be selected from a group consisting of: Dobutamine, Isoprenaline, and Noradrenaline. Preferably, the agonist is Isoprenaline. A suitable β₁-adrenergic receptor agonist may be selected from a group consisting of: Isoprenaline and Salbutamol.

The invention also provides in a fifth aspect, a process for making the composition according to the fourth aspect, the process comprising contacting a therapeutically effective amount of an adrenergic receptor agonist and a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the adrenoceptor agonist is any amount which, when administered to a subject, is the amount of medicament or drug that is needed to treat liver disease/damage or produce the desired effect.

For example, the therapeutically effective amount of adrenergic receptor agonist used may be from about 0.01 mg to about 800 mg, and preferably from about 0.01 mg to about 500 mg. It is preferred that the amount of adrenoceptor agonist is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the adrenoceptor agonist) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The adrenoceptor agonist according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The adrenoceptor agonist may be prepared as a sterile solid composition that may be dissolved or suspended at the time of administration using sterile water, saline, or other appropriate sterile injectable medium.

The adrenoceptor agonist and pharmaceutical compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The adrenoceptor agonists according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

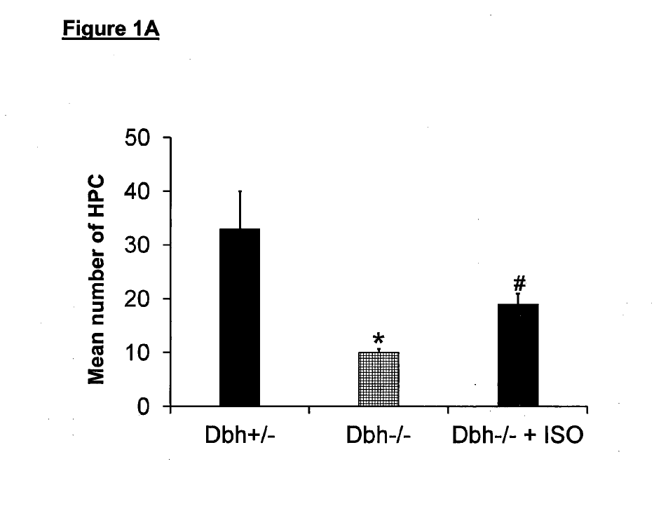

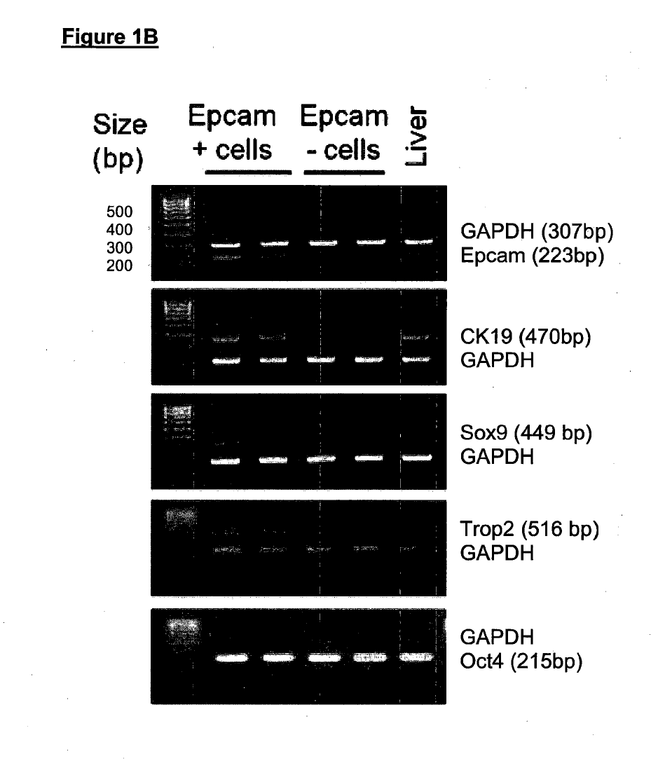

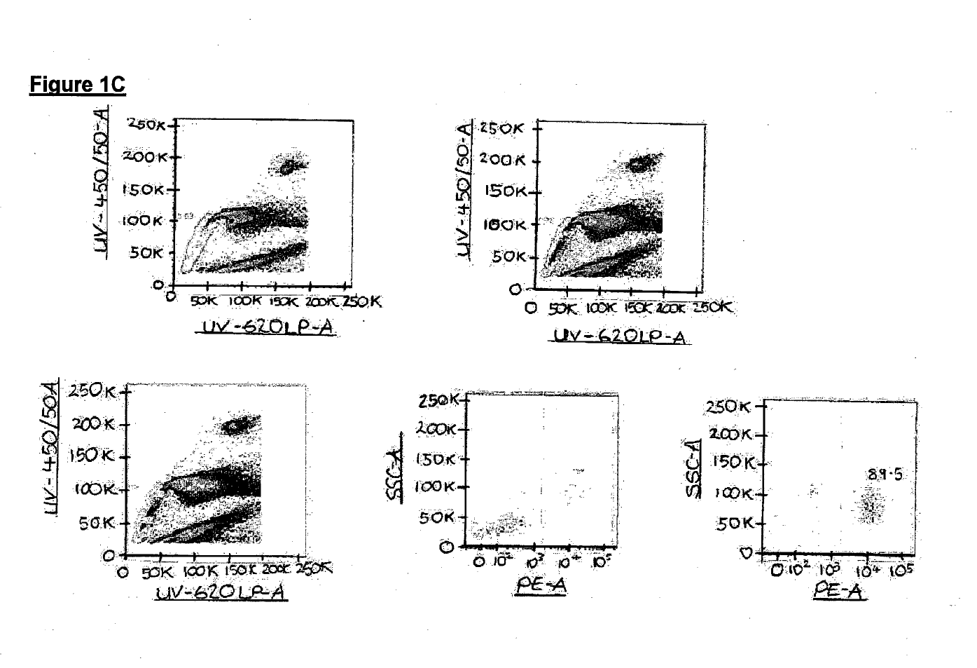

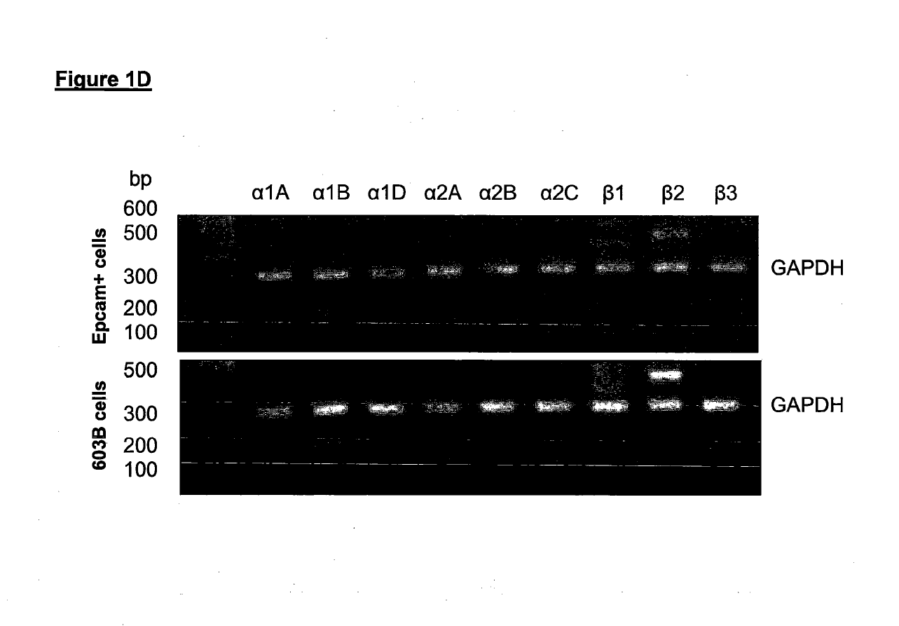

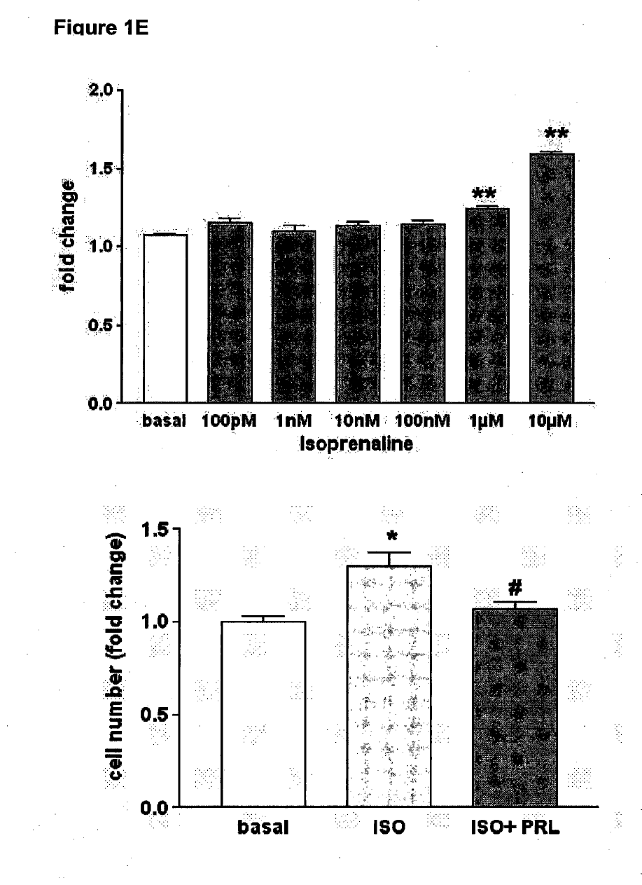

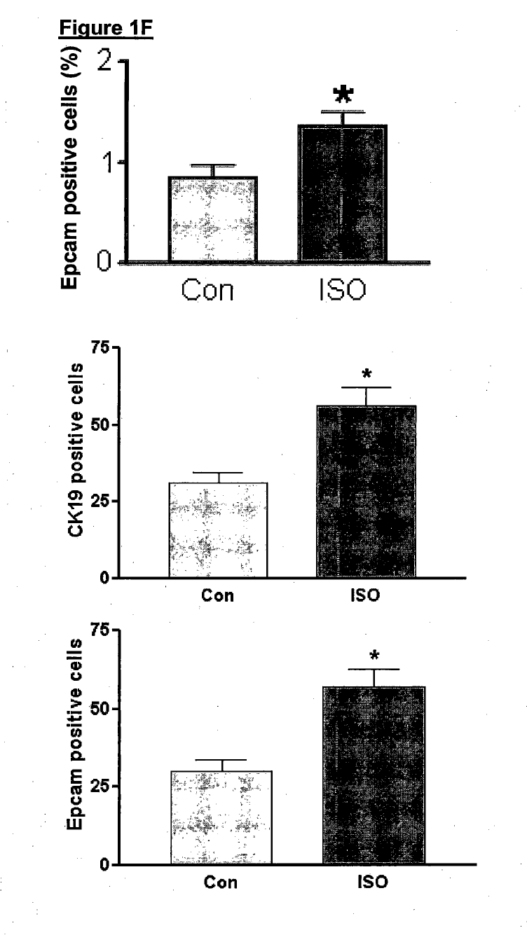

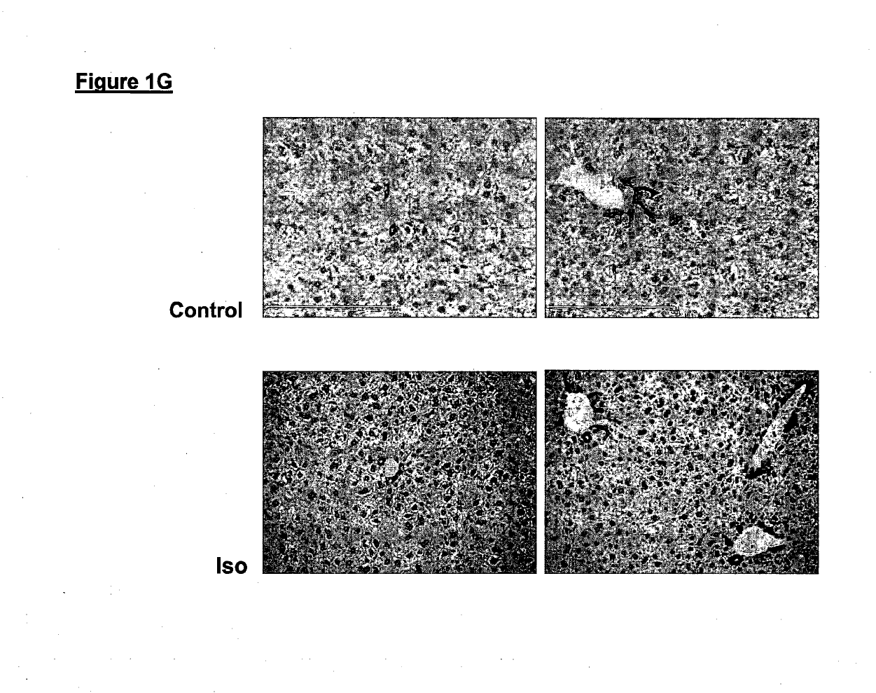

FIG. 1A shows the mean number of CK19 positive HPCs in the liver of mice control Dbh^{+/-}, Dbh^{-/-}, and Dbh^{-/-} mice infused with Isoprenaline (Dbh^{-/-}+ISO) at 20 mg/kg/day to induce activation of the SNS. Data are mean±s.e.m, n=5 mice per group. *p<0.05 in Dbh^{-/-} mice compared to control mice and #p<0.05 in Dbh^{-/-}+ISO compared to Dbh^{-/-} (one-way ANOVA with Tukey's post hoc test);

FIG. 1B shows the results of a duplex PCR performed on isolated EpCAM+ cells (EpCAM+ cells) and EpCAM depleted non-parenchyma cells (EpCAM-cells) from normal mouse liver. Total liver extract served as control;

FIG. 1C are representative flow cytometry plots of side population (SP) cells in total NPC isolated from normal mice liver. The same samples treated with verapamil which inhibit the function of the ABC transporter lost the SP population. EpCAM positive cells were highly enriched in the SP cells. Inset number indicates percentage of positive cells in total NPC;

FIG. 1D shows adrenoceptor mRNA expression of isolated EpCAM+ cells and the liver progenitor cell line (603B cells) using RT-PCR;

FIG. 1E (Left panel) are the results of a cell proliferation assay which show the fold-increase in the number of 603B cells at different doses of isoprenaline (100 pM-10 μM). Results are expressed as fold change±s.e.m from 6 biological replicates relative to control (basal medium). **p<0.001 compared to basal medium control (one-way ANOVA with Tukey's post hoc test);

FIG. 1E (Right panel) are the results of a cell proliferation assay which show the fold-increase in the number of 603B cells treated with basal medium (basal) as control, 10 μM of isoprenaline, and 10 μM of isoprenaline (ISO) after pre-treatment with 10 μM of Propranolol (ISO+PRL). Results are expressed as fold change±s.em, relative to basal from 3 biological replicates; *p<0.05 compared to basal; #p<0.05 compared to ISO;

FIG. 1F shows the percentage of EpCAM+ cells (determined using flow cytometry), the number of CK19 cells (determined using Immunohistochemistry) and the number of EpCAM cells in (the livers of mice) mice which received either a control treatment (Con) or isoprenaline (Iso) at a dose of 2.5 mg/kg. Control mice received PBS vehicle (Con). Data are representative from 2 independent experiments. Data are mean±s.e.m. (n=4 per group);

FIG. 1G are representative images of HPCs detected in mice livers using immunohisotchemistry. Mice received either a control treatment (Con) or isoprenaline (ISO);

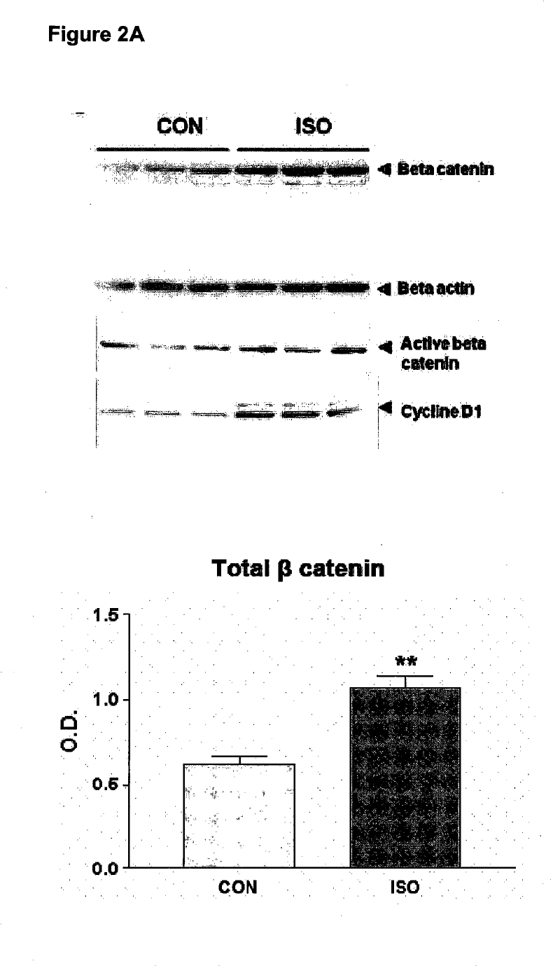

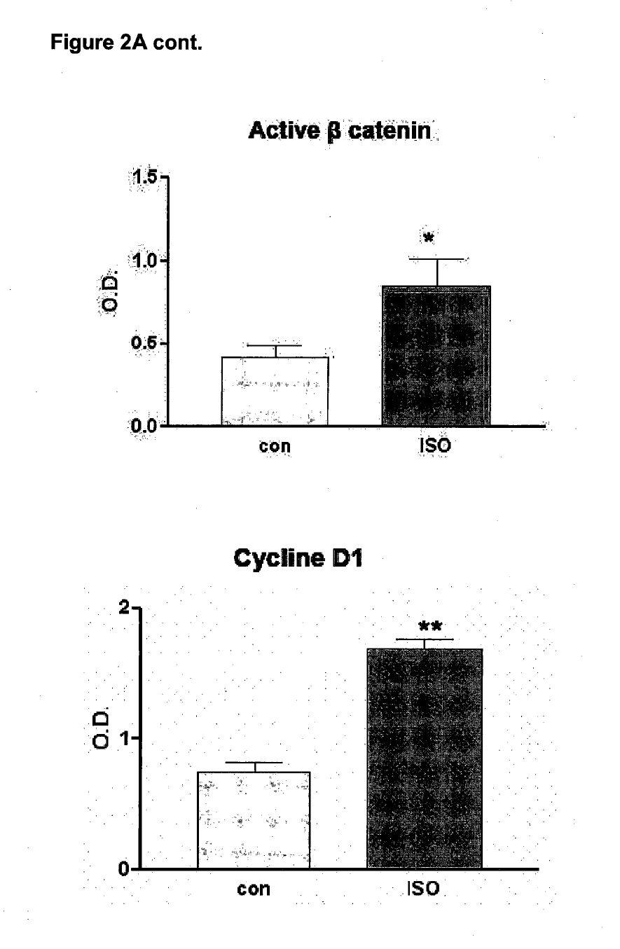

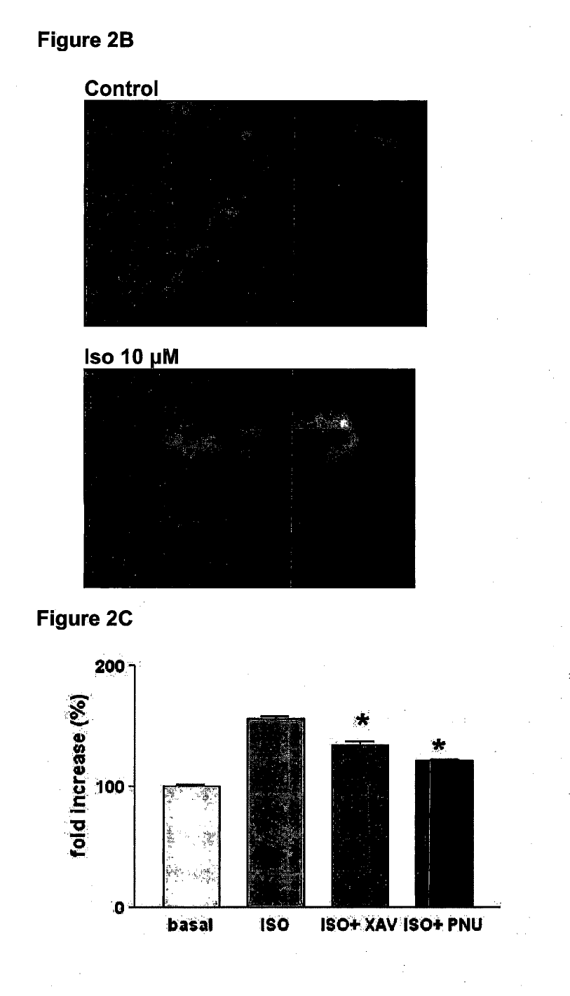

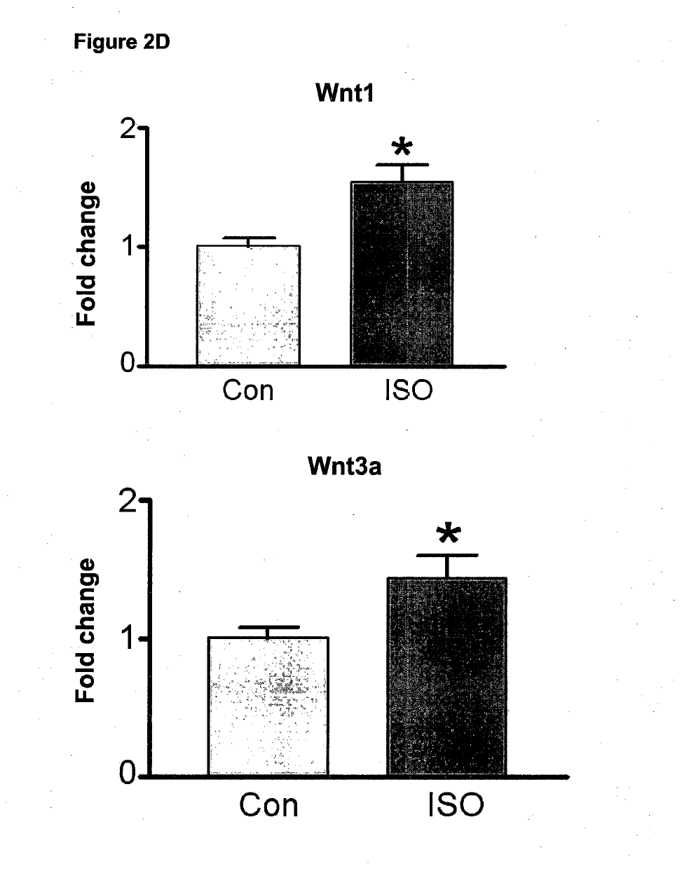

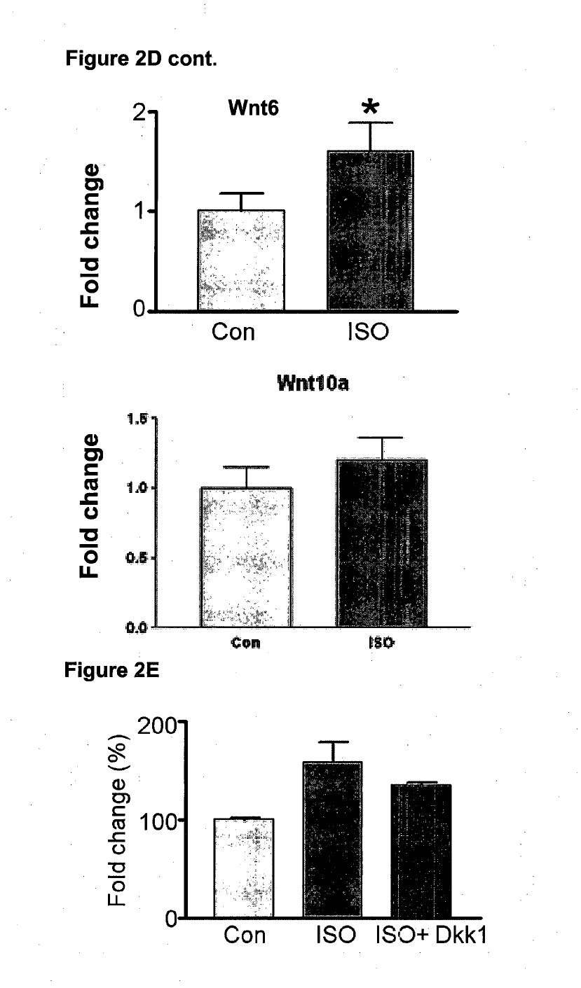

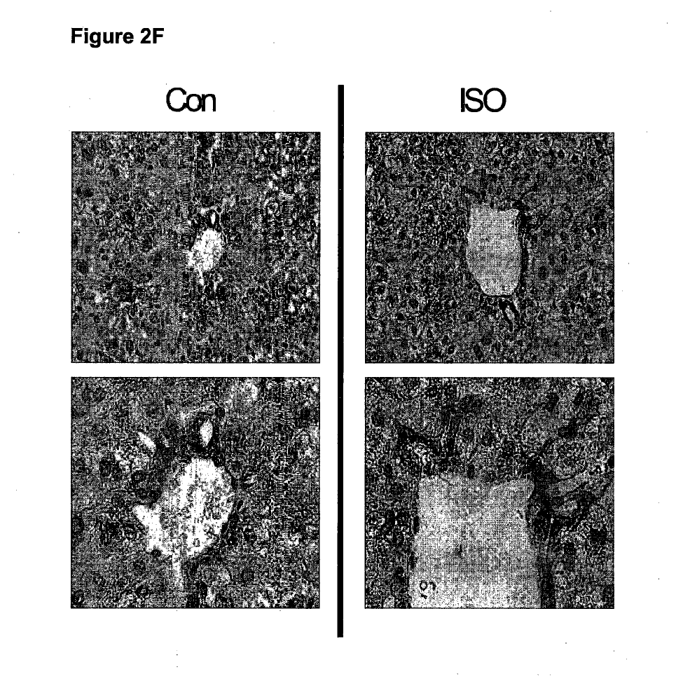

FIG. 2A is a representative western blot and densitometric analyses showing indicated protein expression in 603B cells treated with either 10 μM isoprenaline (ISO) or basal media (Con). An antibody to β-actin was used as a loading control. Data are mean±s.e.m, n=3;

FIG. 2B are immunofluorescent immunocytochemistry images of 603B cells treated with isoprenaline showing cell membrane localisation of β-catenin (left) and nuclear localization of β-catenin (right). Nuclei were stained with dapi (blue);

FIG. 2C is a proliferation assay showing the fold-increase in the number of 603B cells stimulated with 10 μM isoprenaline (ISO) or 10 μM ISO in the presence (or absence) of 1 μIM of the specific Wnt/13 catenin inhibitors XAV939 (XAV) and PNU-74654 (PNU). Data are mean±s.e.m. (n=3); **p<0.001 compared to basal medium; #p<0.05 compared to ISO;

FIG. 2D shows the level of Wnt ligand (Wnt 1, 3a & 6) mRNA expression, assessed by real-time PCR, in 603B cells treated with 10 μM of isoprenaline or control (Con). Data are mean±s.e.m. (n=4), *p<0.05;

FIG. 2E are the results of a CCK8 proliferation assay which show the effect of the Wnt antagonist (recombinant Dkk 1, 0.1 μg/ml) on isoprenaline (10 μM)-stimulated 603B cells. Data are mean±s.e.m (n=3);

FIG. 2F are immunohistochemical stains for active β-catenin in a mouse liver 24 hours after administration of vehicle (Con) or Isoprenaline (ISO). Left panel=lower magnification; Right panel=higher magnification;

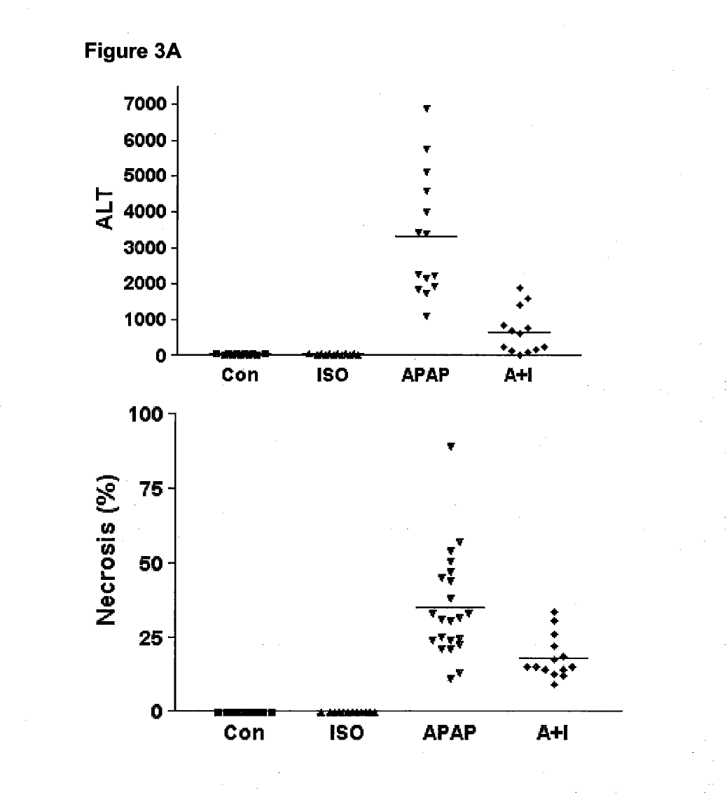

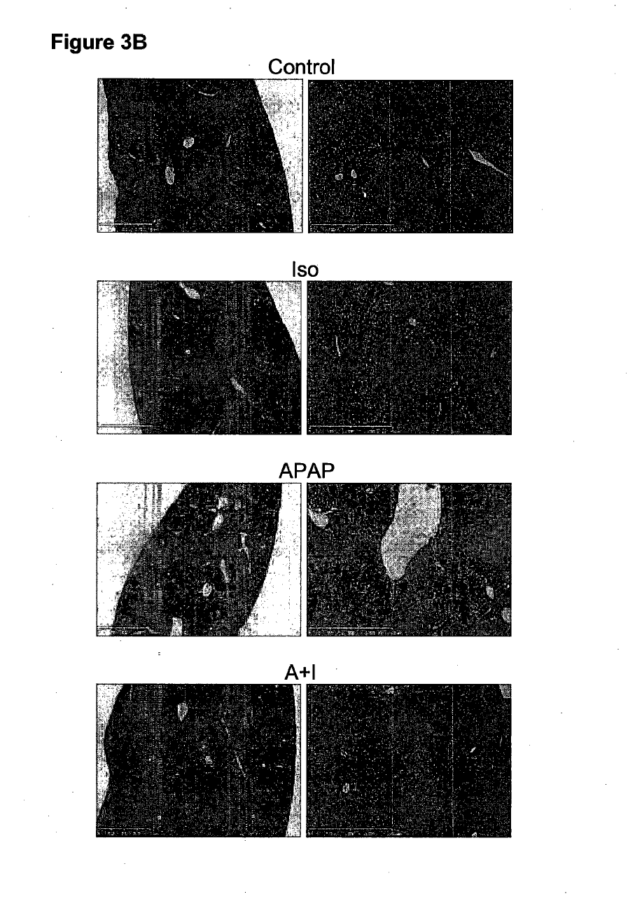

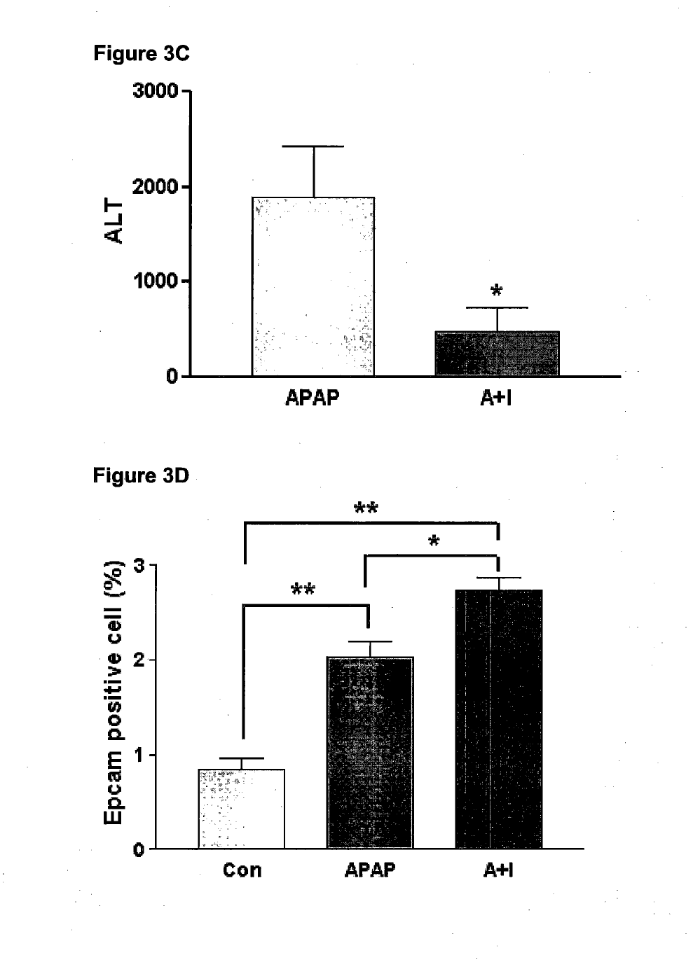

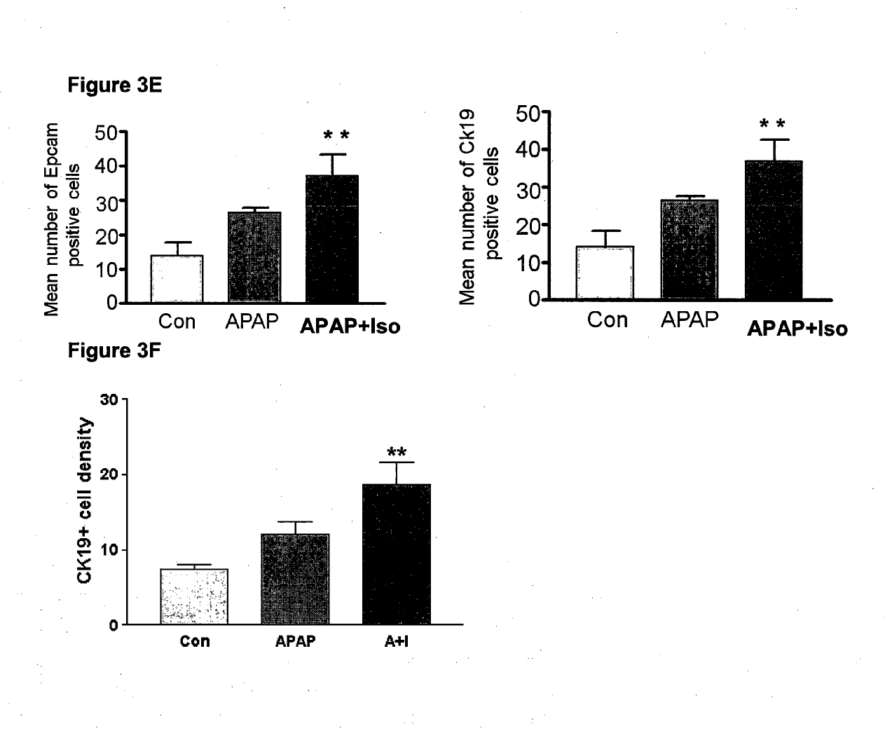

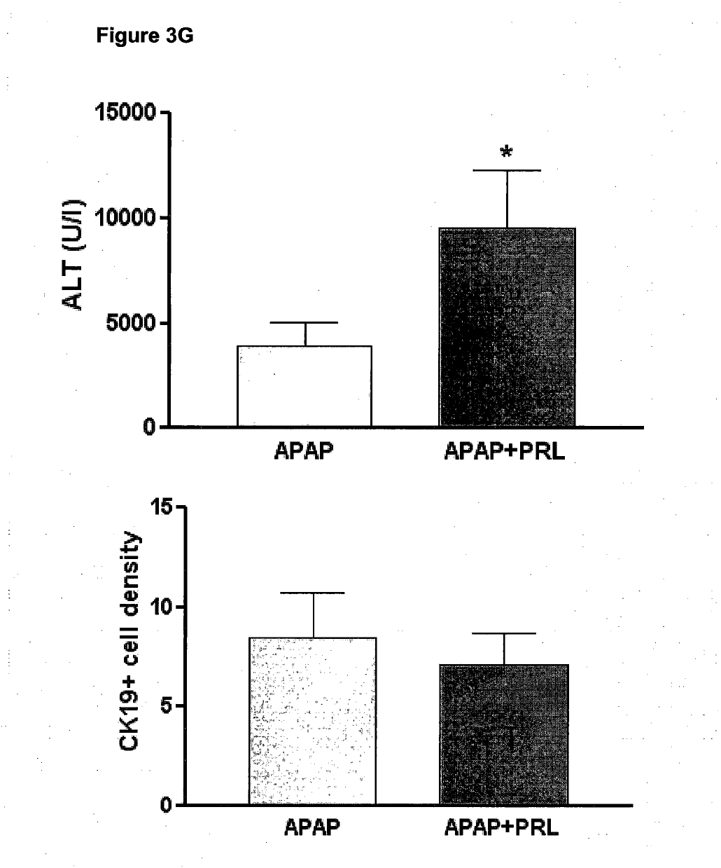

FIG. 3A is a graph showing that ALT (top) and % necrosis (bottom)) are cumulative for all animals either injected with vehicle, isoprenaline (2.5 mg/kg), APAP (375 mg/kg) or APAP with subsequent administration of isoprenaline (A+I) at 24 h after first administration; n=11 to 22 per group;

FIG. 3B is a representative histological image of mice liver 24 hours after injection with either vehicle (control), isoprenaline (2.5 mg/kg), APAP (375 mg/kg) or APAP with subsequent administration of isoprenaline (A+I); left hand panel in each figure is the lower magnification and the right hand panel the higher magnification;

FIG. 3C shows the ALT 3 h of mice after APAP administration. Data are mean±s.e.m, n=4/group; *p=0.05, by 2-tailed unpaired t-test;

FIG. 3D is the flow cytometric analysis of CD45−/EpCAM+ve cells in non-arenchymal cell fraction. Data are mean±s.e.m, n=4/group; *p<0.05, **p<0.001;

FIG. 3E is the immunohisotchemical analyses of progenitor cells using EpCAM (left) and CK19 (right). Data are mean numbers of EpCAM and CK19+ve cells per portal tract±s.e.m (n=5/group);

FIG. 3F shows CK19 positive cell density confined to small portal tracts; *p<0.05, **p<0.001;

FIG. 3G shows liver injury judged by ALT elevation (left) and HPC number determined by CK19 positive cells density (right) in mice treated with APAP alone or APAP with subsequent administration of PRL (10 mg/kg). Data are mean±s.e.m, n=4 each, *p<0.05, ns=not significant;

In FIGS. 3A-3G, Con=vehicle treated, APAP=375 mg/kg of APAP and subsequent vehicle treatment, A+ISO=375 mg/kg of APAP and subsequent 2.5 mg/kg of ISO treatment;

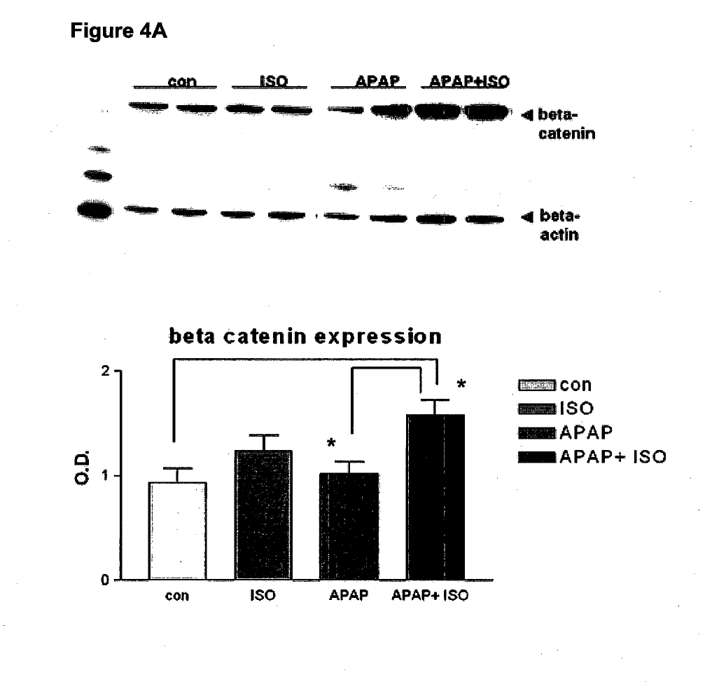

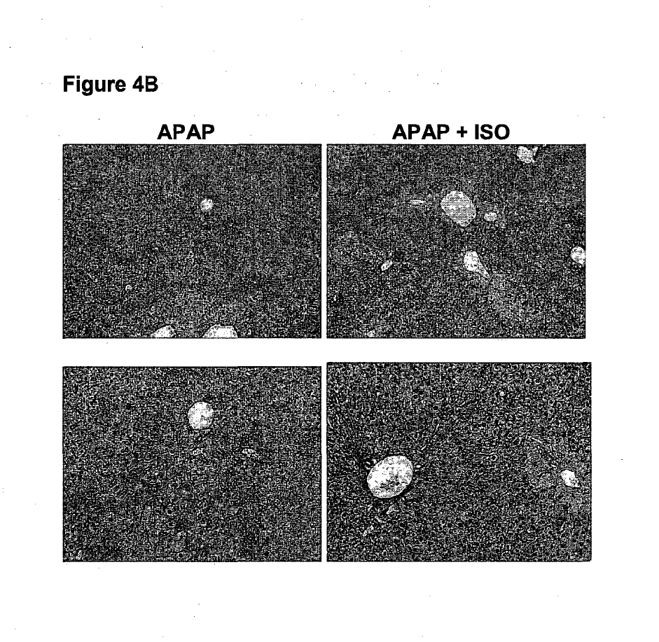

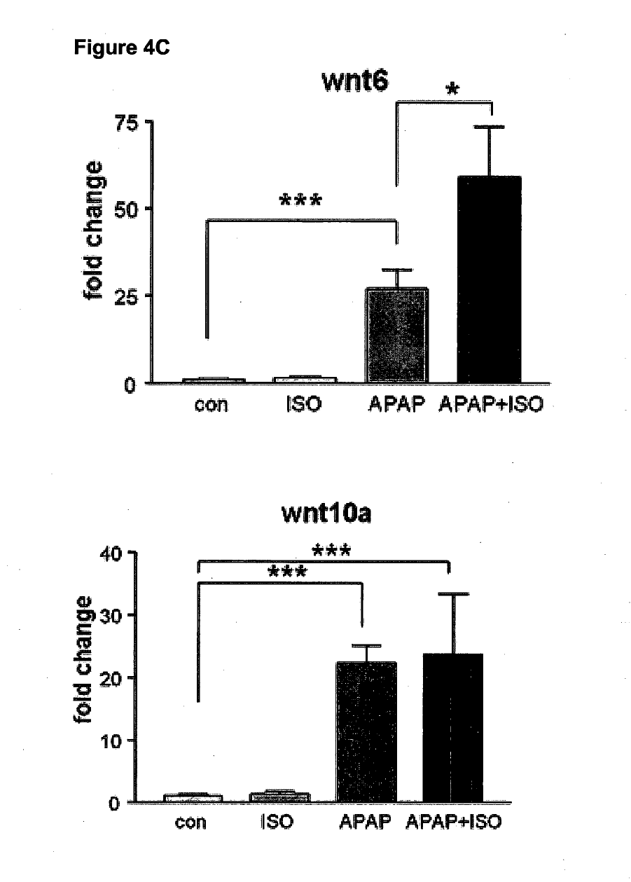

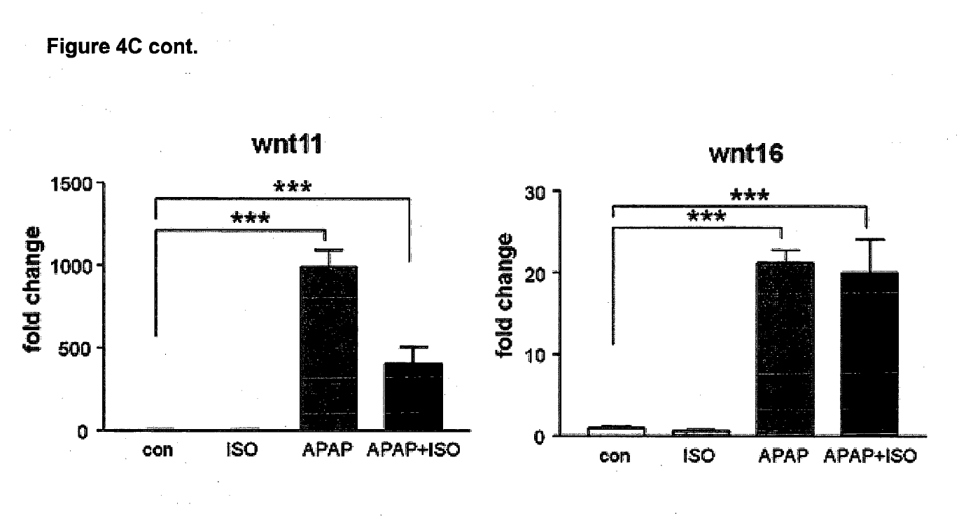

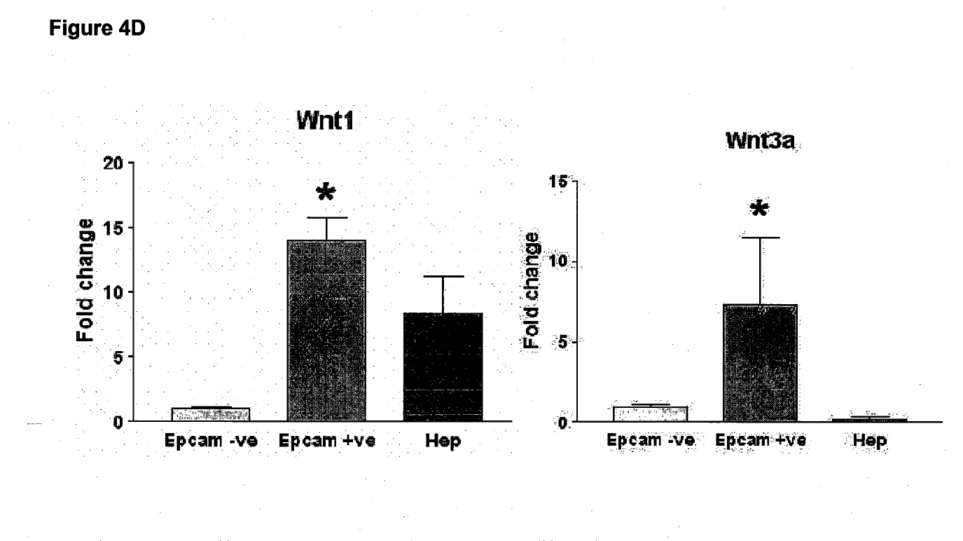

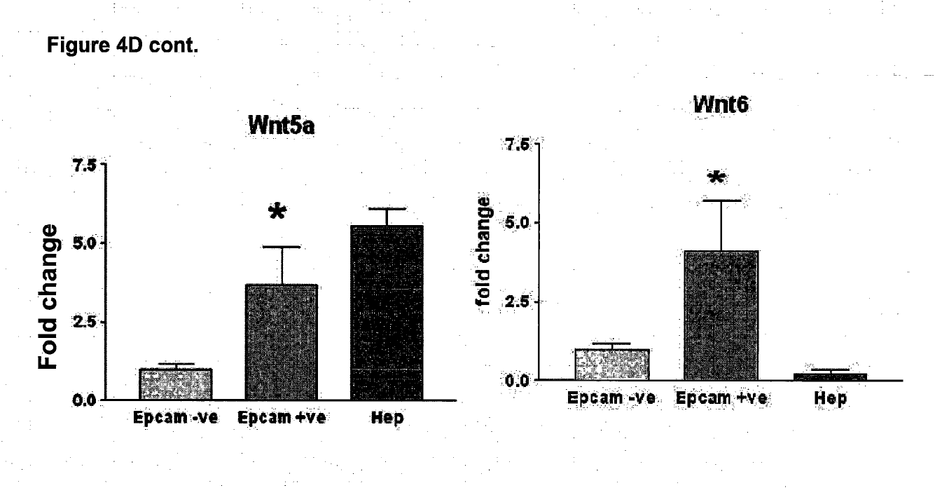

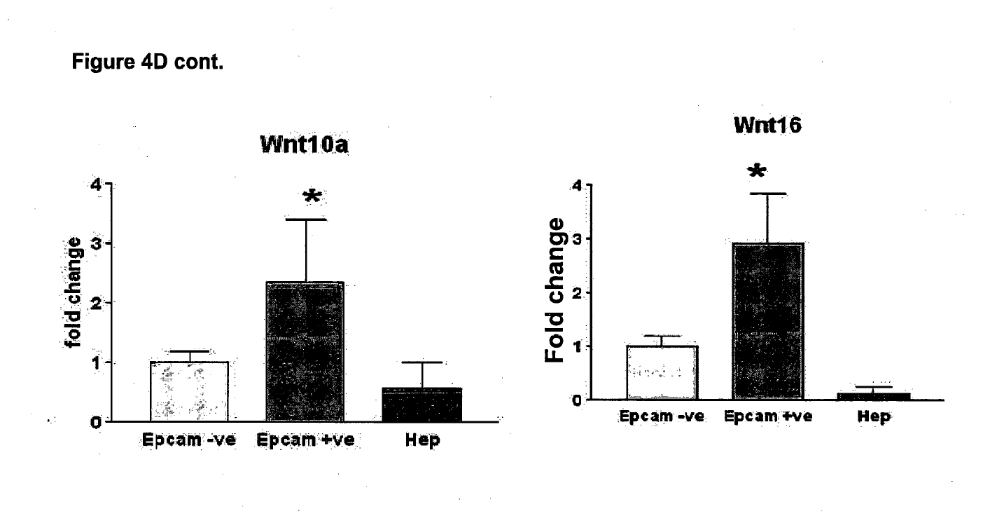

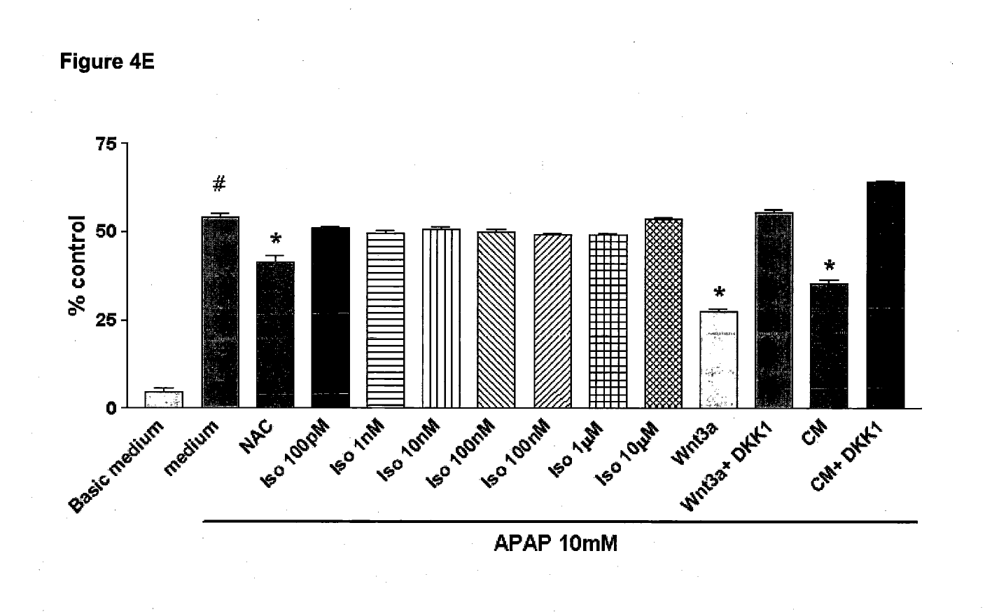

FIG. 4A is a representative western blot (upper) and densitometric (lower) analyses of β-catenin in the livers of mice treated with APAP or APAP+ISO. ISO was administered 1 hour after APAP and the livers were harvested 24 h after APAP initial administration; n=4 per group. *p<0.05;

FIG. 4B are representative micrographs of immunohistochemical staining of active β-catenin in the livers of mice treated with APAP or APAP/ISO (A+I) 24 h after initial administration as in (α) above. Upper panel=lower magnification, Lower panel=higher magnification;

FIG. 4C shows wnt ligand expression in total liver 24 h after initial administration were analyzed in vehicle injected (Con), APAP injected (APAP), and APAP with subsequent ISO injected (A+I) mice. Data are mean±s.e.m, n=4 each. *p<0.05, **p<0.001;

FIG. 4D shows the level of wnt ligand expression in EpCAM+ve cells, the EpCAM depleted non-parenchymal fraction, and hepatocytes isolated from mice livers treated with APAP and ISO 24 h after initial APAP administration;

FIG. 4E are the results of a LDH cell cytotoxicity assay. Isolated hepatocytes from normal mice liver were treated with 10 mM APAP or control medium in the presence or absence of ISO (100 pM-10 μM), 20 mM of N-acethylsysteine (NAC), 100 ng/ml recombinant mouse Wnt3a, Wnt3a with recombinant mouse Dkk1 (0.1 μg/ml), 603B conditioned medium from cells stimulated with ISO (CM) and CM with Dkk1. Each bar represents replicates from 6 wells of the same treatment. Results are expressed as fold change±s.e.m. relative to triton X treated hepatocyte as controls. *p<0.05 compared to APAP alone;

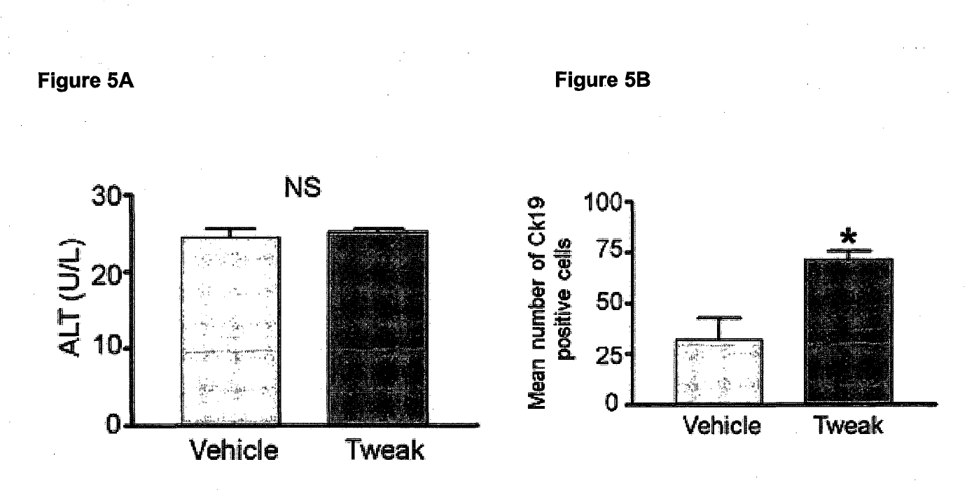

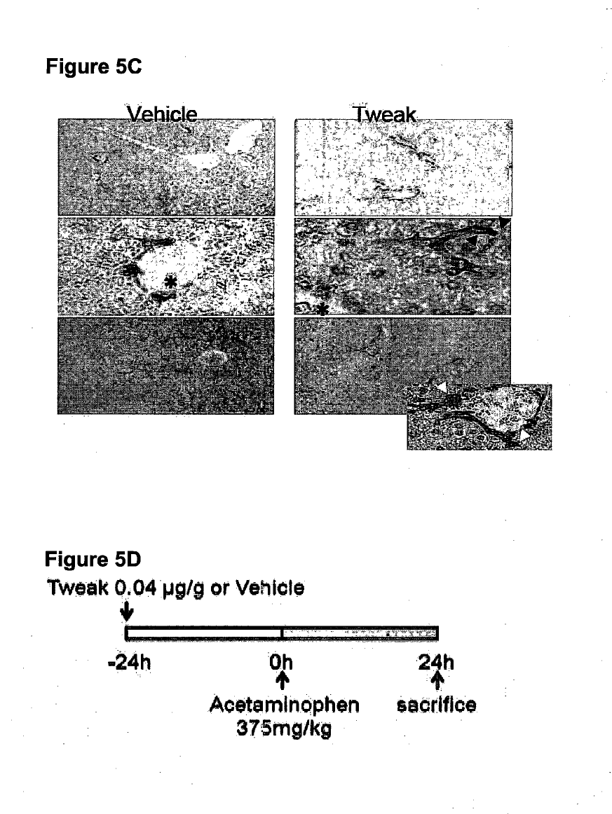

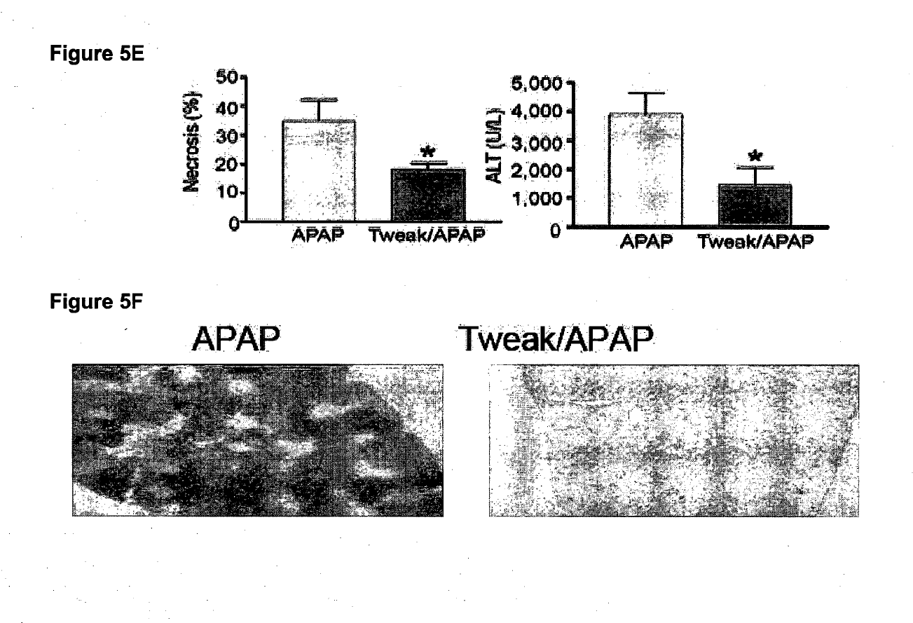

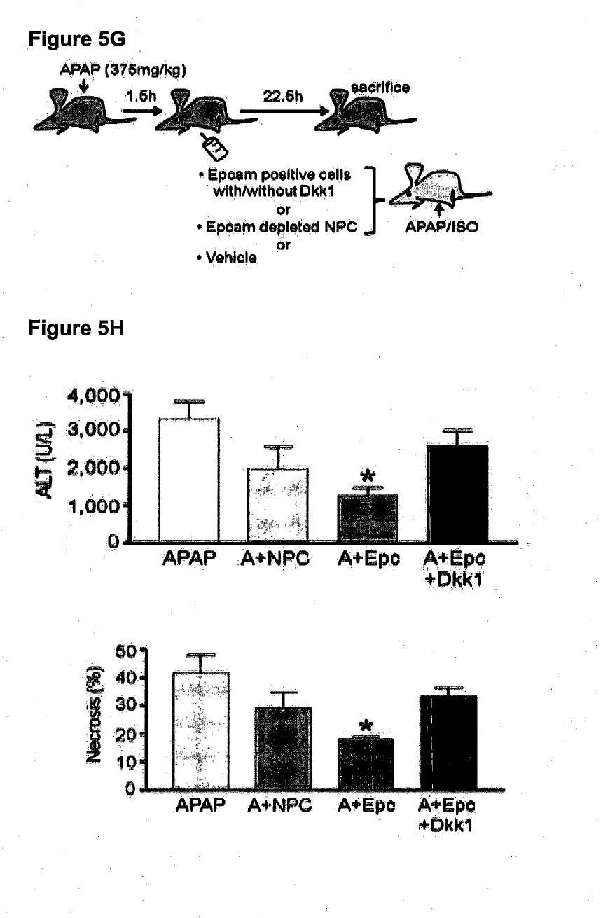

FIG. 5A shows the affect of recombinant TWEAK, 0.04 μg/g on liver injury, assessed using ALT;

FIG. 5B shows the affect of recombinant TWEAK, 0.04 μg/g on CK19+ve HPC cell numbers;

FIG. 5C are representative images of immunohistochemical staining with CK19 (DAB chromogen, brown), upper panels; and CK19 with Ki67 (AEC chromogen, red), double staining (middle panels) and NFκB p65 immunostaining (lower panels). Insert=higher magnification, arrow head indicates positive staining of ki67 in CK 19+ve HPCs. White arrow head indicates nuclear localization of NFKb p65 in periportal ductular cells;

FIG. 5D shows the experimental design of the TWEAK study;

FIG. 5E shows the % necrosis (left) and serum ALT (right) from mice treated with APAP and TWEAK pre-treated mice with subsequent APAP administration. Data are mean'5 s.e.m, n=4 each. *p<0.05, 24 h after initial APAP administration;

FIG. 5F are representative histological images of APAP and TWEAK/APAP mice livers;

FIG. 5G shows the experimental design of EpCAM positive cell administration. Mice were administered APAP. One and half hours later they given EpCAM+ve cells, or EpCAM+ve cells with/without DKK1, EpCAM depleted NPC or vehicle. EpCAM+ve cells were isolated from APAP+ISO treated mice;

FIG. 5H shows the production of the liver injury marker, ALT, 24 h after APAP administration. Serum ALT (left) and % necrosis (right) were analyzed in APAP+ vehicle (APAP), APAP and EpCAM depleted non-parenchymal cells (A+NPC), APAP+EpCAM+ve (A+Epc), and APAP+EpCAM+ve (A+Epc+DKK1). Data are mean±s.e.m, n=4/group. *p<0.05;

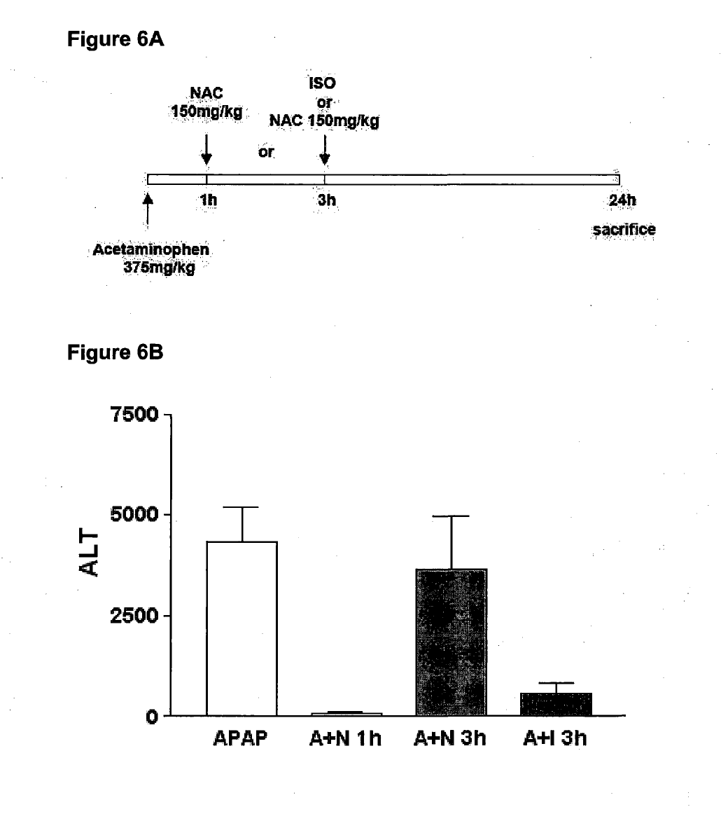

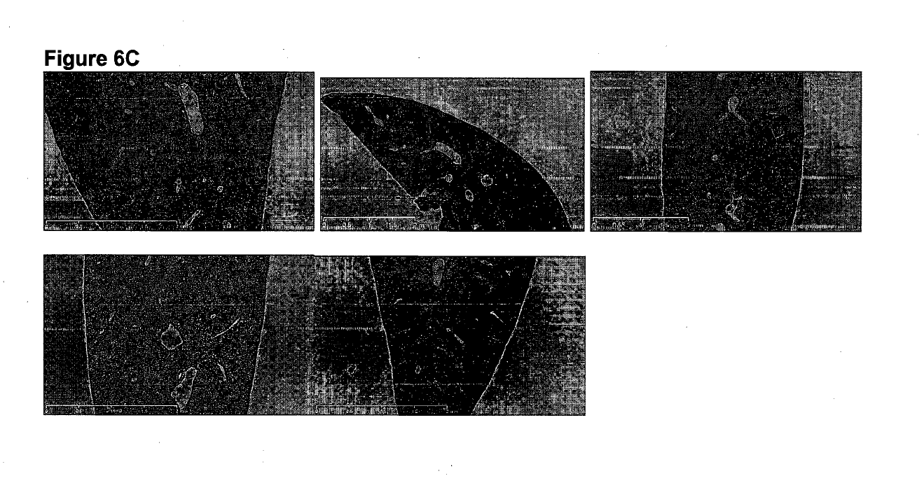

FIG. 6A shows the experimental design of the study used to obtain the results of FIG. 6B;

FIG. 6B shows the ALT of mice which received APAP and 1 or 3 hrs later were received NAC. An alternative batch of mice was treated with APAP followed by ISO 3 hrs after initial APAP;

FIG. 6C shows the immunohistochemical staining of mice which were used in FIG. 6B;

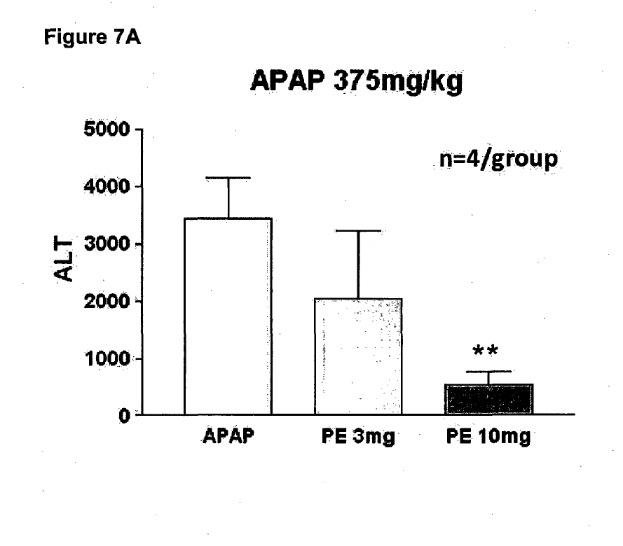

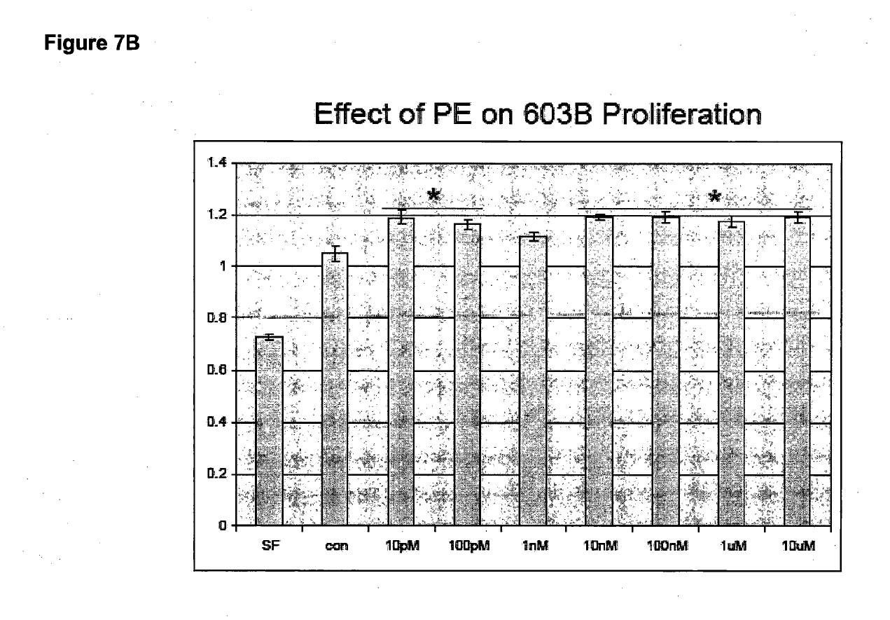

FIG. 7a shows the ALT of mice treated with APAP followed 1 hr later by phenylephrine (PE, 3 mg/kg or 10 mg/kg) and sacrifice 24 hrs after APAP administration; and

FIG. 7b shows the affect of PE (10 pM to 10 μM) on the proliferation of 603B cells. Data are mean±s.e.m. (n=3). **p<0.01 compared to control (Con).

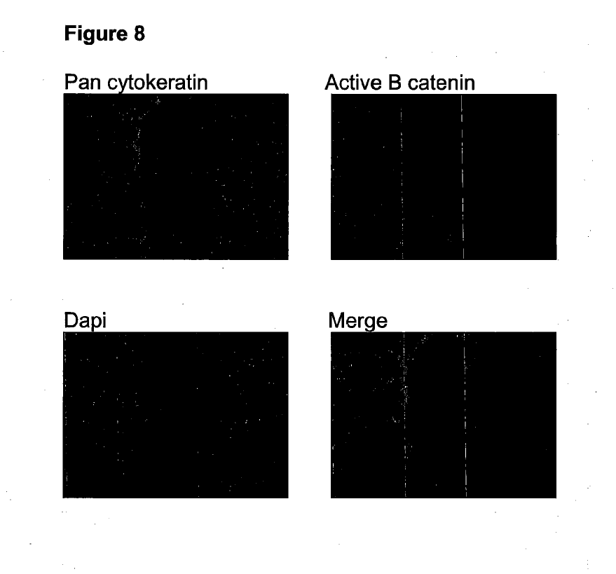

FIG. 8 shows that ISO induces β catenin activation on HPCs in vivo. 10 mg/kg of ISO treated liver were subjected to analysis of active β-catenin (red) expression in pan-cytokeratin positive HPCs (green). Positive β-catenin nuclear staining is as shown on HPCs (yellow).

### MATERIALS & METHODS

### Animals

Male C57BL/6j mice with a mean weight 25 to 30 g were from our Biological Services colony. Male dopamine β-hydroxylase deficient (Dbh^{-/-}) and Dbh^{+/-} mice (30-40 weeks) were also from our colony as previously described (Oben, J. A., et al., 2004). All animals were housed in an environmentally controlled room with 12-h light/dark cycle and allowed free access to food and water. All animals were treated in accordance with The Animals (Scientific Procedures) Act, UK, 1986 guidelines.

### Materials

Culture medium was obtained from Invitrogen. All other chemicals were from Sigma unless otherwise stated.

### Cell Line

Immature murine cholangiocyte cell line (603B cells) were a kind gift from Professor Diehl.

### Animal Experiments

All mice are fasted overnight before APAP administration. APAP was dissolved in warm phosphate buffered saline (PBS) and administered intra-peritoneally (IP) with APAP at a dose of 375 mg/kg, 500 mg/kg or PBS as control. One hour after APAP injection, either Propranolol in water (4 mg/kg), Isoproterenol (ISO) in water (2.5 mg/kg) or water were administered IP. 24 hours after APAP treatment, mice were sacrificed with carbon dioxide. Dbh^{-/-} mice were administered ISO as previously described (Mackintosh, C. A., et al. 2000). Mouse recombinant TWEAK (R&D systems) was administered IP at 0.04 μg/g body weight.

### Cell Isolation

Hepatocytes were isolated as previously reported (Schwabe, R. F., et al., 2001). Hepatic stellate cells, Kupffer cell and hepatic sinusoidal lining cell were extracted by optiprep gradient and subsequent selective adherence method as previously reported (Oben, J. A., et al., 2004; Li, Z., et al. 2002; and Williams, J. M., et al. 2010). Purity of HSC, KC, SEC was assessed by immunocytochemistry using GFAP, aSMA, F4/80 and vWF antibody and revealed 98%, 92%, 87% purity respectively. EpCAM+ cell were isolated by BD Magnet according to the manufacturer's instructions.

### Assessment of Liver Injury

The degree of liver injury was assessed by histology and serum ALT. All liver sections were stained with haematoxylin and eosin (H&E) and scanned by NanoZoomer (Hamamatsu, Japan). Necrotic area was measured and expressed as a percentage of necrotic tissue in whole area of liver section using NDP.view (Hamamatsu, Japan).

### Cell Culture Experiments

603B cell were cultured as previously described (Omenetti, A., et al 2009). For proliferation assay, FBS was reduced to 1% and used as a basal state. LSEC were cultured on collagen coated plate and other liver cell fractions were cultured on normal dish using RPMI-1640 containing 10% FBS. Primary hepatocytes were cultured on either collagen coated 96 well plate or 60 mm dish using Williams E medium supplemented with 10% FBS, insulin-transferrin-selenium G cocktail and 100 nM dexamethasone. After 4 hours plating, cells were washed and replaced with basic media containing the reagents and incubated for a further 2 hours. After 2 hour of reagents treatment, APAP containing media adjusted to 10 mM of final concentration was added. Concentration of the drugs we used in this experiment was decided on the basis of preliminary experiments (data not shown).

### Proliferation Assay

### CCk-8 Assay

Cell proliferation assay was performed using the Cell counting Kit-8 (CCK-8) according to the manufacturer's protocol.

### Direct Cell Counts

To further confirm the CCK-8 assay we also directly counted the cell numbers in some experiments. Adherent cells were treated with 0.25% trypsin solution containing 0.02% EGTA in Ca²⁺ and Mg²⁺ free phosphate-buffered saline at 37° C. for 5 min, and the viable cell number and dead cell number was determined using Nucleocounter (Chemometec).

### Cytotoxic Assay

LDH released from cells were assessed by LDH assay kit (Cayman) with 0.1% Triton X treated cells as positive controls.

### Immunohistochemistry (IHC)

Formalin-fixed paraffin-embedded tissue were cut at 4 μm onto glass slides coated with poly-l-lysine. For chromogenic IHC, antibody binding was visualized using the ImmPRESS Peroxidase Polymer Detection Reagents (Vector lab, UK). For double chromogenic IHC, microwave heat treatment in citric based solution (Vector lab, UK) were applied after the first color development. For immunofluorescence IHC or immunocytochemistry, Alexa Fluor 555 and Alexa Fluor 488 conjugated secondary antibody were used. Nuclei were stained with DAPI (Vector). All images were captured using a Nikon Eclipse e600 microscope and camera (DXM1200F) and acquired with NIS-Elements Advance software (Nikon). HPC numbers were counted by an expert liver pathologist unaware of the identity of the groups as previously described (Oben, J. A., et al. 2003).

### PCR and Semi-Quantitative Real Time PCR

RNA was isolated using TRIzol (Invitrogen), according to the manufacturer's instructions. cDNA was synthesized with the Qiagen QuantiTect Reverse Transcription kit (Qiagen).

Duplicate PCR reactions were performed with multiplex PCR kit (Qiagen) using mixed primer (GAPDH and target primer). Semi-quantitative real time PCR was done with Rotor-Gene 3000 (Corbett Robotics) and QuantiFast SYBR Green PCR kit (Qiagen). All real-time PCR reactions were performed in triplicate with GAPDH as an internal control. Target gene levels in treated samples are presented as a ratio to levels detected in corresponding control samples, according to the ΔΔCt method.

### Western Blotting

Western blotting was performed as described (Soeda, J et al., 2012). Western blots shown are representative of 2 or 3 independent repeats. Semi-quantitative analysis of western blots by densitometry was carried out using LabWorks 4.6 software (UVP, USA).

### Flow Cytometry

Total NPC were extracted and analysed as previously described (Okabe, M., et al. 2009; Yovchev, M. I., et al. 2008; and Lin, K. K. and M. A. Goodell, 2011). Hoechst3332 staining was performed as described (Lin, K. K. and M. A. Goodell, 2011; and Goodell, M. A., et al., 1996) with minor modifications. Briefly, total NPC were adjusted to 10⁶ cells/ml in pre-warmed RPMI complete media (10% FBS, P/S, galutamate), incubated for 90 minutes at 37 degree with 5 ug/ml of Hoechst with verapamil as (50 uM) control. Samples were then washed with ice-cold PBS and incubated with Fc blocker (Cd16/32 mouse monoclonal antibody:BD) and stained with PE conjugated EpCAM (Biolgend) and Alexa-fluor 700 conjugated CD45. Data were analyzed by FlowJo software (version and company).

### Statistical Analyses

All data were expressed as mean±s.e.m. and means were compared by the Student's t-test or ANOVA as appropriate. Sample size per group, n=/>₃ per group.

### EXAMPLE 1

### The SNS Regulates Hepatic Progenitor Cell (HPC) Expansion

To determine if the SNS regulates HPC expansion, it was first confirmed whether Dbh^{-/-} (Dopamine β-hydroxylase) mice, which are genetically deficient in the SNS neurotransmitters norepinephrine (NE) and epinephrine, have a significantly attenuated HPC population compared to their heterozygote controls. HPC populations were enumerated by the immunohistochemical presence of CK-19. As shown in FIG. 1a, treatment with isoprenaline (ISO), a non-specific β-adrenoceptor agonist, significantly recovered HPC numbers in Dbh^{-/-} mice.

To confirm the expression of adrenoceptors on HPCs, EpCAM+ve cells were isolated from the livers of control C57BL mice. Expression of EpCAM (epithelial cell adhesion molecule) has been shown to be a reliable marker of HPCs in mice (Schmelzer, E., et al 2007; Tanaka, M., et al. 2009; Okabe, M., et al. 2009; Yovchev, M. I., et al 2007). These EpCAM+ve cells expressed other known HPC markers, for example CK19, Sox9, TROP2, and Oct4, as shown in FIG. 1b. The EpCAM+ve cells also showed Hoechst 33342 extruding properties, i.e. they were side population (SP) cells, as shown in FIG. 1c. Moreover, these HPCs, expressed α1b-, α1c-, α2α-, α2b-, α2c-, plus β1- and β2-adrenergic receptor subtypes at the mRNA level, as show in FIG. 1d.

The above results were corroborated by double immunofluorescent staining with pan-cytokeratin (another accepted HPC marker (Yin, L., et al 2002)) and β1 and β2 adrenoceptor. This confirmed that β1 and β2 adrenoceptors are expressed on HPCs at the protein level. Therefore, there is an association between the expansion of HPC populations and the SNS, mediated via adrenoceptor.

To further delineate the role of adrenergic stimulation in HPC proliferation, the 603B cell line was used. 603B cells, like HPCs, are derived from the terminal branches of the biliary tree (Ueno, Y., et al 2003; Omenetti, A., et al 2007). As shown in FIG. 1d, 603B cells possess the same adrenoceptor profile as isolated EpCAM+ve cells. This finding validated their further use in this study. Treatment with ISO induced 603B cell proliferation and their pre-treatment with the β-adrenoceptor antagonist, propranolol (PRL), inhibited ISO induced proliferation, as shown in FIG. 1e. Surprisingly, ISO also increased the number of the HPC cells in normal C57BL/6J mice, as determined by expression of EpCAM (flow cytometry) and CK19 (immunohistochemistry), as shown in FIG. 1f and FIG. 1g. Therefore, these findings suggested direct expansion of HPCs by ISO in murine liver.

### EXAMPLE 2

### β-Adrenoceptor Stimulation Activates the Canonical Wnt Pathway on HPCs

To elucidate the molecular pathway through which stimulation of β-adrenoceptors induces proliferation of HPCs, the canonical Wnt pathway was investigated. As shown in FIG. 2a, total β-catenin expression was significantly increased in ISO treated 603B cells. Expression of dephophorelated β-catenin (activated β-catenin) and cyclin D1 which is a known β-catenin target gene were also significantly upregulated in ISO treated 603B cells. Furthermore, immunofluorescence cytochemistry showed accumulation of β-catenin in the nuclei of ISO treated 603B cells, as shown in FIG. 2b. This data suggests that ISO treatment activated the canonical Wnt pathway in 603B cells.

To further elucidate the molecular pathway through which stimulation of β-adrenoceptors induces proliferation of HPCs, the effect of β-catenin specific inhibitors were used in proliferation assays with 603B cells. As shown in FIG. 2c, ISO-induced proliferation was partially but significantly inhibited by β-catenin specific inhibitors. This indicates that the effect of ISO on 603B proliferation is partly mediated by β-catenin. ISO treatment also significantly increased Wnt1, 3a, 6 and ma mRNA expression in 603B cells, as shown in FIG. 2d. These Wnt ligands are known to activate the canonical Wnt pathway (Koch, S., et al 2011) and thus suggest that ISO-induced 603B proliferation is partly autocrine in nature.

To confirm the findings of Example 6 (above), the Wnt antagonist DKK1 (Koch, S., et al 2011) was used in the presence of ISO. FIG. 2e shows that there was a trend towards statistical difference between the proliferation of ISO-treated and ISO plus DKK1-treated 603B cells.

The effect of β-adrenoceptor stimulation on the canonical Wnt pathway was studied further in vivo. As shown in FIG. 2f, mice treated with ISO showed upregulation of Wnt6 mRNA in total liver at 24 h after injection and strong β-catenin immunoreactivity on periportal ductular cell detected by immunohistochemistry. Double immunofluorescence confirmed these cells were HPCs (see FIG. 8). These results indicate that ISO treatment also activates the canonical Wnt pathway on HPC in vivo.

### EXAMPLE 3

### ISO Protects Against APAP-Induced Liver Injury and Enhances HPCs Expansion

To determine whether above-mentioned findings have any relevance with respect to liver disease, an APAP induced liver injury model which results in massive hepatic necrosis and progenitor cell proliferation was used (Williams, C. D. et al 2011; Kofman, A. V., et al 2005).

Mice were initially administered APAP at 500 mg/kg intraperitoneally. This resulted in a significant number of deaths, and was reduced by ISO treatment. Therefore, the dose of APAP was reduced to 375 mg/kg. 1 h after administration with APAP, mice were treated with either ISO or PBS vehicle. As shown in FIGS. 3a and 3b, APAP treatment induced massive hepatic necrosis as judged by histology and ALT 24 h after APAP treatment. ISO treatment significantly reduce the ALT (3332±462.9 vs. 674.1±173 IU/L, p<0.0001) and hepatic necrosis (350.25±3.745 vs. 18.48±1.935%, p<0.0001). FIG. 3C shows that a significant elevation in ALT was detected as early as 3 h after APAP treatment, and this elevation in ALT was significantly attenuated by treatment with ISO.

The number of HPCs in the livers of the various treatment groups was analyzed using flow cytometry and immunohistochemistry. As shown in FIGS. 3d and 3e, ISO treatment significantly increased the number of HPCs even though injury, as shown in FIG. 3c, was far less compared to the APAP alone group. The density of HPC in the smallest portal tract, was also analyzed by CK19 positivity, as it is reported that in APAP induced liver injury models the density of CK19 positive cells in the smallest portal tract is a more precise quantification compared to the absolute number. FIG. 3f shows that the HPC density was significantly increased in the APAP+ISO group compared to the APAP alone group. To clarify the significance of β-adrenoceptor signalling in this model, the β-adrenoceptor antagonist PRL was used. FIG. 3g shows that PRL treatment markedly increased injury and resulted in reduced numbers of HPCs.

### EXAMPLE 4

### Expanding Hepatic Progenitor Cells are the Main Source of Wnt

The inventors then decided to determine how ISO treatment protects the liver from APAP induced injury. In order to do this, the canonical Wnt pathway was investigated. Canonical Wnt signalling is reported to be hepatoprotective against APAP induced liver injury in addition to its role in HPC proliferation.

Mice were initially administered APAP at 500 mg/kg intraperitoneally. This resulted in a significant number of deaths, and was reduced by ISO treatment. Therefore, the dose of APAP was reduced to 375 mg/kg. 1 h after administration with APAP, mice were treated with either ISO or PBS vehicle. As shown in FIG. 4a, Western blotting showed that β-catenin expression was significantly increased in the livers of ISO treated mice compared to those treated with APAP alone and controls at 24 h after injection. Immunohistochemistry using activated β-catenin antibody also showed strong β-catenin staining in the livers of APAP+ISO treated groups. Analysis of 16 known various Wnt ligands in the APAP and APAP+ISO treated livers showed that Wnt6, Wnt10a, Wnt11 and Wnt16 were upregulated in the APAP alone and APAP+ISO groups, with Wnt6 showing significantly higher expression in the APAP+ISO group compared to APAP only group, see FIG. 4c. At this time, significant HPCs expansion was also detected in the livers of APAP+ISO mice. Importantly, Wnt 6, 10a, 11 and 16 were significantly upregulated in APAP and APAP+ISO group at 2 h after APAP administration. Among these Wnt ligands, Wnt 10a showed significantly higher expression in the APAP+ISO group. Moreover, significant β-catenin activation in the APAP+ISO group was also detected at 3 h after APAP administration. These data suggested that ISO treatment enhanced the canonical Wnt pathway.

To further define the cell types in the liver responsible for these ligand upregulation, hepatocytes, EpCAM positive cells, and EpCAM depleted non-parenchymal cells were isolated from the livers of mice treated with APAP+ISO. Wnt ligand expression in these fractions was then analyzed. As shown in FIG. 4d, among the Wnt ligands which was upregulated in vivo, the EpCAM positive cell fraction showed significant higher Wnt6, 10a, and 16 expression compared to the other fractions. In addition, the EpCAM positive fraction showed the highest expression of Wnt1 and Wnt3a. Among these upregulated Wnt ligands, Wnt1, 3a, 6 and 10a are known to induce the canonical Wnt pathway.

To evaluate the possible influence of ISO induced Wnt upregulation on liver cells, Wnt expression in the various liver cell types in the presence and absence of ISO was analyzed ex vivo. Wnt 6 expression was detected in isolated hepatic stellate cells (HSC) and Kupffer cells (KC). Culture activation significantly upregulated Wnt 6 expression in HSC compared to freshly isolated HSC. However, ISO did not induce upregulation of Wnt 6 in HSC or KC. These data suggest that the major source of Wnt is HPCs. The inventors also investigated several cytokines which can induce HPC proliferation but they could not detect any ISO specific significant elevation in these cytokines. These results suggested that ISO treatment increases HPC number as well as their expression of Wnt to subsequently activate the canonical Wnt pathway in hepatocytes and protect from APAP toxicity.

To further support this postulate, primary hepatocytes were extracted from mice livers and treated with APAP. This treatment significantly induced their death as judged by release of LDH. ISO pre-treatment did not protect the hepatocytes from APAP induced death at any dose investigated. However, recombinant Wnt3a pre-treatment significantly protected the hepatocytes against APAP. As shown in FIG. 4e, the conditioned media from 603B stimulated with ISO significantly protected the hepatocytes, an effect reversed by recombinant DKK1. Western blotting showed that ISO did not increase β-catenin expression on hepatocytes but rWnt3a and ISO stimulated conditioned media induced increased β-catenin expression (data not shown). These results strongly suggested that ISO protects hepatocytes from APAP induced cell death not directly but through paracrine activation of the canonical Wnt pathway.

### EXAMPLE 5

### Hepatic Progenitor Cell Expansion is Hepatoprotective

The above Examples indicate that expanding HPCs are the source of Wnt and that these HPCs have a protective role in APAP induced liver injury. To test this hypothesis, Tumour associated weak inducer of apoptosis (TWEAK) was used together with direct HPC administration. TWEAK has been reported to specifically promote progenitor cell expansion in the liver with no effect on hepatocytes (Jakubowski, A., et al 2005). To take advantage of this property, recombinant TWEAK was administered before APAP treatment and expansion of the endogenous HPCs.

As shown in FIGS. 5a and 5b, TWEAK administration induced HPC proliferation without any evidence of hepatocyte cell death, as judged by ALT and active caspase-3 immunostaining. The effect of TWEAK is mediated by NF-kB signaling in HPCs (Tirnitz-Parker, J. E., et al 2010) and NF-kB p65 immunostaining has revealed a strong cytoplasmic and nuclear expression of the protein, especially in periportal ductular cells, see FIG. 5c. Furthermore, expansion of HPC by TWEAK administration protected from APAP induced liver injury, as shown in FIGS. 5d, 5e and 5f.

As described in FIG. 5g, pooled EpCAM positive cells were from the livers of mice treated with APAP+ISO 2 h after APAP administration were then administered to mice which has been treated with APAP. EpCAM positive cells injection significantly ameliorated liver injury compared to vehicle and EpCAM depleted non-parenchymal cells. DKK1 treatment reversed the effect of EpCAM positive cell administration, as shown in FIG. 5h.

### EXAMPLE 6

### Delayed Administration of Isoprenaline (ISO)

The effects of ISO in combination with the current gold-standard treatment, NAC, were also investigated. FIG. 6A shows the experimental design of the study. As shown in FIG. 6B, administration of 150 mg/kg of NAC markedly reduces hepatocyte injury when administered 1 hour following overdose. However, NAC did not have a protective role if it was administered 3 hrs post APAP. Conversely, ISO markedly reduced APAP induced-liver injury even at 3 hrs post APAP.

### EXAMPLE 7

### Effect of the α1-Adrenoceptor Phenylephrine Agonist on APAP Induced Liver Injury

To determine if the α₁-adrenoceptor agonist, phenylephrine, induces effects similar to isoprenaline, the protocol of Example 16 (above) was repeated with ISO.

APAP was administered at 375 mg/kg and either phenylephrine (PE) or PBS vehicle were given 1 h after APAP. As shown in FIG. 7a, APAP alone induced substantial liver injury reflected by increased ALT (3500±750). This effect was moderately reduced by PE 3 mg (2000±1200) and significantly reduced by PE 10 mg (450±200, p<0.005).

To investigate the pathways through which PE protects from APAP induced liver injury, 603B cells were cultured in the presence and absence of PE with ISO. As shown in FIG. 7B, it was found that PE, at 10 pM to 10 μM, induced moderate but significant proliferation of 603B cells.

### REFERENCES

Hiramoto, T., Ihara, Y. & Watanabe, Y. alpha-1 Adrenergic receptors stimulation induces the proliferation of neural progenitor cells in vitro. Neurosci Lett 408, 25-28 (2006).
Kim, M. O., Na, S. I., Lee, M. Y., Heo, J. S. & Han, H. J. Epinephrine increases DNA synthesis via ERK1/2s through cAMP, Ca(2+)/PKC, and PI3K/Akt signaling pathways in mouse embryonic stem cells. J Cell Biochem 104, 1407-1420 (2008).
Schmelzer, E., et al. Human hepatic stem cells from fetal and postnatal donors. J Exp Med 204, 1973-1987 (2007). 19. Tanaka, M., et al. Mouse hepatoblasts at distinct developmental stages are characterized by expression of EpCAM and DLK1: drastic change of EpCAM expression during liver development. Mech Dev 126, 665-676 (2009).
Okabe, M., et al. Potential hepatic stem cells reside in EpCAM+ cells of normal and injured mouse liver. Development 136, 1951-1960 (2009).
Yovchev, M. I., Grozdanov, P. N., Joseph, B., Gupta, S. & Dabeva, M. D. Novel hepatic progenitor cell surface markers in the adult rat liver. Hepatology (Baltimore, Md. 45, 139-149 (2007).
Yin, L., Lynch, D., Ilic, Z. & Sell, S. Proliferation and differentiation of ductular progenitor cells and littoral cells during the regeneration of the rat liver to CCl4/2-AAF injury. Histol Histopathol 17, 65-81 (2002).
Ueno, Y., et al. Evaluation of differential gene expression by microarray analysis in small and large cholangiocytes isolated from normal mice. Liver Int 23, 449-459 (2003).
Omenetti, A., et al. Hedgehog-mediated mesenchymal-epithelial interactions modulate hepatic response to bile duct ligation. Lab Invest 87, 499-514 (2007).
Koch, S., et al. The Wnt antagonist Dkk1 regulates intestinal epithelial homeostasis and wound repair. Gastroenterology 141, 259-268, 268 e251-258.
Williams, C. D., Koerner, M. R., Lampe, J. N., Farhood, A. & Jaeschke, H. Mouse strain-dependent caspase activation during acetaminophen hepatotoxicity does not result in apoptosis or modulation of inflammation. Toxicology and applied pharmacology 257, 449-458 (2011).
Kofman, A. V., et al. Dose- and time-dependent oval cell reaction in acetaminophen-induced murine liver injury. Hepatology (Baltimore, Md. 41, 1252-1261 (2005).
Jakubowski, A., et al. TWEAK induces liver progenitor cell proliferation. The Journal of clinical investigation 115, 2330-2340 (2005).
Tirnitz-Parker, J. E., et al. Tumor necrosis factor-like weak inducer of apoptosis is a mitogen for liver progenitor cells. Hepatology (Baltimore, Md. 52, 291-302 (2010).
Oben, J. A., et al. Hepatic fibrogenesis requires sympathetic neurotransmitters. Gut, 53(3), 438-45 (2004).
Mackintosh, C. A., et al. Overexpression of antizyme in the hearts of transgenic mice prevents the isoprenaline-induced increase in cardiac ornithine decarboxylase activity and polyamines, but does not prevent cardiac hypertrophy. Biochem J, 350 Pt 3, 645-53 (2000).
Li, Z., et al. Murine leptin deficiency alters Kupffer cell production of cytokines that regulate the innate immune system. Gastroenterology, 123(4), 1304-10 (2002).
Williams, J. M., et al. The role of the Wnt family of secreted proteins in rat oval “stem” cell-based liver regeneration: Wnt1 drives differentiation. Am J Pathol. 176(6), 2732-42 (2010).
Omenetti, A., et al. Repair-related activation of hedgehog signaling promotes cholangiocyte chemokine production. Hepatology, 50(2), 518-27 (2009).
Oben, J. A., et al., Sympathetic nervous system inhibition increases hepatic progenitors and reduces liver injury. Hepatology, 2003. 38(3), 664-73 (2003).
Soeda, J., et al., Nicotine induces fibrogenic changes in human liver via nicotinic acetylcholine receptors expressed on hepatic stellate cells. Biochem Biophys Res Commun, 41 7(1), 17-22 (2012).
Yovchev, M. I., et al. Identification of adult hepatic progenitor cells capable of repopulating injured rat liver. Hepatology, 47(2): p. 636-47 (2008).
Lin, K. K. and M. A. Goodell. Detection of hematopoietic stem cells by flow cytometry. Methods Cell Biol, 103: p. 21-30 (2011).
Goodell, M. A., et al., Isolation and functional properties of murine hematopoietic stem cells that are replicating in vivo. J Exp Med, 183(4), 1797-806 (1996).

1-20. (canceled)

21. A method of treating, ameliorating or preventing liver damage in a subject, the method comprising administering, to a subject in need of such treatment, a therapeutically effective amount of an adrenergic receptor agonist.

22. The method according to claim 21, wherein the liver damage which is treated is acute liver damage.

23. The method according to claim 21, wherein the liver damage is caused by administration or consumption of a poison, for example paracetamol, alcohol, or Khat plant.

24. The method according to claim 21, wherein the agonist is a β-adrenergic receptor agonist.

25. The method according to claim 21, wherein the adrenergic receptor agonist is a β₁-, a β₂- or a β₃-adrenergic receptor agonist.

26. The method according to claim 25, wherein the β₁-adrenergic receptor agonist is selected from a group consisting of Dobutamine, Isoprenaline, and Noradrenaline.

27. The method according to claim 25, wherein the β₁-adrenergic receptor agonist is Isoprenaline.

28. The method according to claim 25, wherein the β₂-adrenergic receptor agonist is selected from a group consisting of Isoprenaline and Salbutamol.

29. The method according to claim 21, wherein the agonist is either an α₁ or an α₂-adrenergic receptor agonist.

30. The method according to claim 29, wherein the α₁-adrenergic receptor agonist is selected from a group consisting of Noradrenaline, Xylometazoline, Phenylephrine, and Methoxamine.

31. The method according to claim 29, wherein the α₂-adrenergic receptor agonist is selected from a group consisting of Clonidine, Dexmedetomidine, Medetomidine, and Romifidine.

32. The method according to claim 21, wherein the agonist is operable, in use, to enhance HPC expansion, preferably by activating the Wnt pathway.

33. A method for inducing the expression of Wnt by hepatic progenitor cells, the method comprising contacting a hepatic progenitor cell with an adrenergic receptor agonist.

34. The method according to claim 33, wherein expression of Wnt 1, 3a, 6 or 10a is induced by the agonist compared to the level of expression in the absence of the agonist.

35. A liver damage treatment composition, comprising an adrenergic receptor agonist and a pharmaceutically acceptable vehicle.

36. A composition according to claim 35, wherein the agonist is selected form a group consisting of Dobutamine, Isoprenaline, and Noradrenaline.

37. A composition according to claim 35, wherein the composition comprises liver-targeting means, arranged, in use, to target the adrenoceptor agonist at least adjacent the liver.

38. A process for making the composition according to claim 37, the process comprising contacting a therapeutically effective amount of an adrenergic receptor agonist and a pharmaceutically acceptable vehicle.
